(19) United States
(12) Patent Application Publication
(10) Pub. No.: US 2022/0175842 A1
(43) Pub. Date: Jun. 9, 2022

(54) EXOSOMES AND USES THEREOF IN DISEASES OF THE BRAIN

(71) Applicants: Darwin J. PROCKOP, Philadelphia, PA (US); Ashok K. SHETTY, Austin, TX (US); Qianfa LONG, Xi'an (CN); Dinesh UPADHYA, Karnataka (IN); Bharathi HATTIANGADY, San Diego, CA (US); Dong-ki KIM, Temple, TX (US)

(72) Inventors: Darwin J. PROCKOP, Philadelphia, PA (US); Ashok K. SHETTY, Austin, TX (US); Qianfa LONG, Xi'an (CN); Dinesh UPADHYA, Karnataka (IN); Bharathi HATTIANGADY, San Diego, CA (US); Dong-ki KIM, Temple, TX (US)

(21) Appl. No.: 16/346,806

(22) PCT Filed: Nov. 2, 2017
(86) PCT No.: PCT/US2017/059787

§ 371 Date: May 1, 2019

Related U.S. Application Data
(60) Provisional application No. 62/416,638, filed on Nov. 2, 2016.

(51) Int. Cl. A61K 35/28; A61P 25/00; A61K 9/127

(52) U.S. Cl. CPC A61K 35/28; A61K 9/127; A61P 25/00

(57) ABSTRACT

Disclosed are medicaments and methods for inhibiting brain inflammation and the cognitive memory loss attendant brain disease and damage in an animal. Status epilepticus, stroke and Alzheimer's disease are particular pathologies that are treated employing pharmaceutically acceptable preparations of the A1 exosomes. The preparations comprise an enriched population of A1 exosomes, such as exosomes derived from culture medium from mesenchymal stem cells. Medicaments and methods for inhibiting pattern recognition and/or memory impairment attendant a brain injury event or degenerative brain disease are also disclosed, comprising administering a pharmaceutically acceptable preparation of exosomes, particularly A1 exosomes, that are CD9− and that prevent the elevation of pro-inflammatory cytokines attendant a brain injury or disease.

### CROSS-REFERENCE TO RELATED APPLICATIONS

The present application claims priority to U.S. Provisional Application 62/416,638, filed Nov. 2, 2016. The entire contents of said application are specifically incorporated herein in its entirety.

### FIELD OF THE INVENTION

This invention relates to the field of brain diseases associated with inflammation, such as status epilepticus and Alzheimer's disease. The invention also relates to the field of treatment medicinal preparations and methods for treating and/or inhibiting brain diseases associated with inflammation, such as treatment preparations comprising exosomes and/or extracellular vesicles.

### BACKGROUND OF THE INVENTION

It is now generally recognized that one means whereby cells communicate is to secrete vesicles in which various cargos are enclosed in a membrane. The vesicles, generally referred to as exosomes or extracellular vesicles (EVs), constitute a special class of small vesicles (about 100 nM in diameter), that lack many of the proteins found on the surface of the cells that secrete them. Some of the cargos of exosomes are bound to the surface of the vesicles. This presents a serious problem in purifying exosomes for therapeutic uses, since these cargos are readily lost during most procedures used to purify exosomes.

Exosomes of various forms have been described relating to treatment of disease. For example, human adipose tissue-derived mesenchymal stem cells contain neprilysin, which degrades amyloid-.β, a pathogenic protein of Alzheimer's disease. When exosomes secreted by human adipose tissue-derived mesenchymal stem cells were administered to the brains of Alzheimer's disease model mice, the generation of amyloid-β was reportedly inhibited (US Pub 20140341882).

Numerous conditions involve damage to the brain, including head trauma, stroke, brain tumors, brain infections, and Alzheimer's disease, and can cause seizures (such as status epilepticus (SE)), stoke, as well as inflammatory and infectious diseases.

Epilepsy and Seizures: Epilepsy is diagnosed when an individual experiences repeated convulsions over a given period of time (Oby, E and Janigro D., 2006, Epilepsia, 47:1761-1774). Not always involving convulsions, seizures are episodes of abnormal electrical activity in the brain which can manifest as changes in attention or behavior. Common causes of epilepsy include congenital brain defects, infections, stroke, traumatic brain injury (TBI), metabolic disorders and brain tumors (van Vliet E A, et al., 2007, Brain, 130: 521-534). A correlation exists between disruption of the blood-brain barrier (BBB) and seizures. Analysis of small molecular tracers has shown that these tracers enter the brain when the BBB is disrupted in both human and animal studies of epilepsy (van Vliet E A, et al., 2007, Brain, 130: 521-534). Furthermore, abnormal electroencephalogram (EEG) patterns can be observed when there is hyper-permeability of the BBB. Serological studies in patients with epilepsy have shown the presence of neuronal and glial proteins (that normally are not present in the blood) as a consequence of BBB deregulation in epilepsy. While several methods can be used to determine directly the proper function of the BBB in animal models, techniques for evaluating BBB integrity in humans have not been reported.

According to one definition, SE is a constant or near-constant state of having seizures. SE is a health crisis that requires immediate treatment. The time point at which treatment is given to a patient has been highly correlated with recovery rate. SE is not very well characterized, and no definitive standard of treatment for SE exists.

Stroke: Stroke denotes a sudden disruption or stoppage of blood flow in the brain which subsequently deprives brain tissue of oxygen and nutrients. The interruption in blood flow can occur as a result of a blood clot blockage (ischemic stroke) or rupture (hemorrhagic stroke) of a cerebral blood vessel. (Lo E H, Dalkara T and Moskowitz M A., 2003, Nat Rev Neurosci., 4:399-415). After the onset of stroke, edema formation develops and induces a rise in intracranial pressure which can lead to compression, hemiation and damage of brain tissue. Increase in cerebrovascular permeability due to BBB disruption is a critical factor in the development of edema (Jiang Q et al., 2005, J. Cereb Blood Flow Metab., 25:583-592). Often the edema that forms worsens during the phase of reperfusion. Inflammatory mediators and cellular proteins from injured cells activate the endothelium and augments BBB permeability contributing not only to edema formation but to the disruption in neuronal homeostasis (Cipolla M J, Huang Q and Sweet J G., 2011, Stroke, 42:3252-3257).

Inflammatory and Infectious Diseases of the Brain: Many infectious diseases affecting the brain cause changes to the brain vasculature that often lead to a breach of the blood brain barrier (BBB). Examples of these types of diseases include viral infections caused by HIV-1, Rabies, cerebral malaria, and Japanese encephalitis virus. (Persidsky, Y et al. 1997, J Immunol, 158:3499-3510; Fabis M J, Phares T W, Kean R B, Koprowski H and Hooper D C. 2008, Proc Natl Acad Sci USA., 105:15511-15516; Tripathi A K, Sha W, Shulaev V, Stins M F and Sullivan D J, Jr. 2009, Blood, 114: 4243-4252; Liu T H, Liang L C, Wang C C, Liu H C and Chen, W J., 2008, J. Neurovirol., 14: 514-521). Also bacterial infections caused by E. coli K1, group B. streptococcus, L. monocytogenes, C. freundii and S. pneumonia strains have been shown to affect the BBB. (Huang S H, Stins M F and Kim K S., 2000, Microbes Infect, 2:1237-1244). Under inflammatory conditions, the normal function of the BBB is compromised due to overproduction of pro-inflammatory molecules by inflammatory cells.

Whether induced by trauma (i.e TBI), cerebrovascular accident (stroke), a pathogen or neurological disorder (i.e multiple sclerosis, Alzheimer's disease), the breach of the BBB is significantly driven by the up-regulation of inflammatory pathways in activated cells of the neurovascular unit and by the recruitment of immune cells (Persidsky Y and Ramirez S H. In The Neurology of AIDS (Gendelman H E, et al., eds) pp. 220-230. Oxford University Press, New York). BBB disruption is markedly enhanced by the recruitment of immune cells to the brain endothelium in a process that involves immune adhesion and transendothelial migration. Therefore, BBB injury in neuroinflammation is considered to at least in part result from the disruption of junction complexes between brain microvascular endothelial cells that facilitate the diffusion of blood products and entry of leukocytes into the brain parenchyma.

The hippocampus of the brain is especially vulnerable to detrimental effects in a subject suffering status epilepticus (SE), Alzheimer's disease, or stroke. During and after the SE event, the brain evidences a series of morphological and functional changes that causes cognitive and mood dysfunction and chronic epilepsy associated with greatly waned neurogenesis (Hattiangady et al., 2004, 2010; Ben-Ari, 2012; Kleen et al., 2012; Loscher et al., 2012; Sankar et al., 2012). Early changes such as loss of glutamatergic neurons, gamma-amino butyric acid (GABA)-ergic inhibitory interneurons (Ben-Ari, 2012), increased oxidative stress, inflammation typified by reactive astrocytes and activated microglia (Fellin and Hydon, 2005; Vezzani et al., 2011), abnormal neurogenesis exemplified by anomalous migration of newly born neurons and greatly waned neurogenesis in the chronic phase have been of some interest (Parent et al., 1997; Shetty and Hattiangady 2007; Scharfman et al., 2012), as these changes can contribute to cognitive and mood dysfunction, as well as the development of chronic epilepsy after SE.

Antiepileptic drug (AED) therapy can stop SE in some instances, but cannot adequately suppress the multiple SE-induced detrimental changes described above (Loscher et al., 2013; Temkin, 2001, 2004; Dichter, 2009). Consequently, AED therapy has mostly failed to prevent the evolution of SE into cognitive and memory impairments and a chronic epileptic state.

The medical arts remains in need of medicaments and methods for containing and/or inhibiting brain inflammation and the brain damage associated with inflammation. Such would provide more effective approaches to inhibiting, reducing and/or preventing cognitive and/or recognition memory impairment and other symptoms attendant diseases associated with brain inflammation and trauma, including Alzheimer's disease, stroke, TBI, Parkinson's disease, epilepsy, and status epilepticus (SE), as well as related diseases of the brain.

### SUMMARY OF THE INVENTION

The present invention, in a general and overall sense, relates to medicaments and methods for using specific preparations of exosomes, termed A1 exosomes, in a neuroprotective strategy for diseases of the brain associated with blood brain barrier (BBB) damage or trauma. By way of example, such diseases include epilepsy, SE, stroke, Alzheimer's disease, Parkinson's disease, traumatic brain injury (TBI) and related brain diseases. In particular, these medicaments and methods are provided to halt or reduce cognitive and memory impairment.

The methods and preparations described are capable of restraining glutamatergic and GABA-ergic neuron loss, oxidative stress, inflammation and maintaining normal neurogenesis in the brain, especially these events after the occurrence of damage to the brain.

A pharmaceutical preparation comprising elements isolated from a cell culture, such as from a cell culture of stem cells, including a culture medium collected from mesenchymal stem cells (MSCs) and other types of cells (human or non-human), have been identified. These elements are defined herein as exosomes (interchangeably referred to herein as vesicles, especially extracellular vesicles (EVs)). The exosomes, in some embodiments, may be described as a preparation that is enriched for an A1 population or preparation of exosomes. The A1 preparation of exosomes are characterized as being absent a CD9 epitope (CD9-) on their surface, as having a mean size of about 85 nm to about 250 nm (such as between about 85 nm and about 236 nm) and as having an anti-inflammatory cytokine inhibiting activity. In other embodiments, the A1 exosome preparations may be described as comprising exosomes having a mean size of about 85 nm to about 100 nm (monomers), about 160 nm to about 200 nm (such as about 165 nm) (dimers) and/or about 205 nm to about 280 nm (or about 207 nm to about 235 nm) (trimers). Specific AI exosome preparations are provided comprising a population of exosomes having a mean size of about 207+/−1.8 nm, about 216+/−−2.3 nm, and about 231+/−3.2 nm (SEM).

The pharmaceutical preparations may also be described as comprising a population of exosomes having a defined protein content. By way of example, one such population of exosomes, the A1 exosomes, as provided in a therapeutic dose in the preparation, may be described as comprising about 30 μg protein, or up to about 200 μg protein/mL saline (or other physiologically acceptable carrier solution). In some A1 exosome preparations, the protein content may be described as comprising a low amount of about 4 ng of native TSG-6. In other embodiments, the number of A1 exosomes provided in a therapeutic dose of the pharmaceutical preparation may be described as comprising about an A1 exosome number of about 15×10⁹ A1 EVs.

In some embodiments, the A1 exosomes are provided as a pharmaceutical preparation. In particular embodiments, the pharmaceutical preparation may be formulated as an intranasal preparation or as an intravenous preparation, or other type of injectable pharmaceutical preparation. An injectable preparation suitable for injection to the brain may also be provided.

The pharmaceutical formulations comprising the A1 exosome preparations are also characterized as having neuroprotective and anti-inflammatory properties. Formulations of the A1 exosomes are also characterized as inhibiting and/or preventing brain injury induced long-term detrimental effects, especially loss of cognitive function and memory impairment.

In a general and overall sense, a medicament and method for treating diseases of the brain associated with brain inflammation are provided employing the formulations and preparations enriched for the A1 EVs. By way of example, such diseases and/or disease-inducing states include epilepsy, status epilepticus (SE), Alzheimer's disease, Parkinson's disease, traumatic brain injury (TBI), and stroke, among others.

In particular embodiments, a medicament and method of treating a patient having a brain induced injury are provided. In some embodiments, the method comprises inhibiting and/or preventing brain injury induced long term detrimental effects, especially loss of cognitive function and memory impairment, in an animal having suffered a brain induced injury by administration of a formulation comprising A1 exosomes. By way of example, such forms of brain induced injury may be observed in a patient having epilepsy, status epilepticus, stroke, or Alzheimer's disease. In other embodiments, the method may comprise administering a therapeutically effective amount of a formulation enriched for a population of A1 exosomes to the patient as an intranasal formulation.

In another aspect, a medicament and method of reducing neurodegeneration and neuroinflammation in a patient in need thereof, is provided. In some embodiments, the method comprises administering a therapeutically effective dose of an exosome preparation (specifically an A1 exosome preparation by intranasal administration, immediately after a status epilepticus event. The results presented here demonstrate that intranasal administration of a formulation enriched for A1 exosomes prevents and/or inhibits cognitive and memory impairment in an animal having experienced an SE episode.

In one particular embodiment, a method for inhibiting cognitive memory loss in an animal having status epilepticus (SE) disease is provided. By providing a preparation including an A1 exosomes to an animal in need thereof.

In another embodiment, a medicament and method is provided for easing SE-induced glutamatergic and GABA-ergic neuron loss, inflammation, long-term decline in neurogenesis in the hippocampus and memory impairments of an animal, comprising administering a pharmaceutical preparation of A1 exosomes. Administration may be intranasal, intravenous, and/or intracranial.

In yet another embodiment, the medicament and method provides for enhancement of neurogenesis in an animal (such as a human), comprising administering to the animal a therapeutically effective amount of an exosome preparation, such as a preparation of A1 exosomes.

In another aspect, an intranasal preparation for treatment of brain deterioration and/or function associated with a post status epilepticus (SE) event is provided, the preparation comprising A1Exsomes in a therapeutically effective amount.

### BRIEF DESCRIPTION OF THE DRAWINGS

FIG. 1—Exosomes (or extracellular vesicles, EVs) reach the hippocampus within 6 hours after intranasal administration.

FIG. 2 A-FIG. 2N—Intranasal administration of EVs after SE prevents the elevation of multiple pro-inflammatory cytokines and chemokines in the hippocampus. The different pro-inflammatory proteins shown are FIG. 2A—TNF; FIG. 2B—IL-1B; FIG. 2C—MCP-1; FIG. 2D—SCF; FIG. 2E—MIP-1; FIG. 2F—GM-CSF; FIG. 2G—IL-12; FIG. 2H—IL-10; FIG. 2I—G-CSF; FIG. 2J—PDGF-B; FIG. 2K—IL-6; FIG. 2L—IL-2; FIG. 2M—TNF-ELISA; FIG. 2N—IL1-β ELISA. EV administration enhanced the concentration of anti-inflammatory cytokine IL-10. Intranasal administration of A1-exosomes two hours after SE eases inflammation in the hippocampus when examined 24 hours post-SE. Bar charts compare the relative concentrations of multiple cytokines between naïve control animals, animals receiving vehicle after SE (SE+Veh) and animals receiving A1-exosomes after SE (SE+EVs). Assays were by multiplexed ELISAs. Animals in SE+Veh group display increased concentration of pro-inflammatory cytokines TNF-a, IL1-β, MCP-1, SCF, MIP-1a, GM-CSF and IL-12 (A-G) whereas animals in SE+EVs group exhibit significantly reduced concentration of these cytokines. This group also showed increased concentration of anti-inflammatory cytokines and growth factors such as IL-10, G-CSF, PDGF-B, IL-6 and IL-2 (H-L). Bar charts in M and N compare levels of TNF-α and IL1-β in the hippocampus measured through independent enzyme-linked immunoassays. In comparison to naïve controls, the concentrations of these proinflammatory cytokines are increased in the SE+Veh group but normalized in the SE+EVs group. *, p<0.05; **, p<0.01; ***, p<0.001; ****, p<0.0001.

FIG. 3A-FIG. 3C-FIG. 3A—Micorgraphs of glutamatergic neurons in tissues; FIG. 3B—Dentate Hilus; FIG. 3C—Intranasal administration of exosomes after SE prevents loss of glutamatergic neurons in the hippocampus.

FIG. 4A-FIG. 4D—Intranasal administration of exosomes after SE prevents loss of GABA-ergic interneurons in the hippocampus. FIG. 4A—Microgiapts of interneurons in tissue; FIG. 4B—DH&GCL subfield; FIG. 4C—CA1 subfield; FIG. 4D—CA3 subfield.

FIG. 5A-5D—Intranasal administration of exosomes after SE eases inflammation in the hippocampus. Intranasal administration of A1-exosomes two hours after SE greatly reduces the density of ED-1+ (CD68+) activated microglia in the hippocampus when examined 4 days post-SE. FIGS. 5A1-5B3 illustrate the distribution of ED-1+ activated microglia in the dentate gyrus (5A1, 5B1), the CA1 subfield (5A2, 5B2) and the CA3 subfield (5A3, 5B3) of an animal that received vehicle after SE (SE-VEH, 5A1-5A3) and an animal that received A1-exosomes after SE (SE-EVs, 5B1-5B3). DH, dentate hilus; GCL, granule cell layer; ML, molecular layer; SO, stratum oriens; SP, stratum pyramidale; SR, stratum radiatum. Bar charts in 5C-5D compare the numbers of ED-1+ microglia in the dentate gyrus (5C), CA1 and CA3 subfields (5D), and the entire hippocampus (5E). Animals receiving A1-exosomes (SE-EVs group) display reduced numbers of ED-1+ activated microglia compared to animals receiving vehicle (SE-VEH group). Scale bar, 100 μm.*, p<0.05; **, p<0.01.

FIG. 6—Intranasal administration of exosomes after SE prevents object recognition memory impairment. Habitual Phase, SE-VEH Group; SE-EVs Group.

FIG. 7—Intranasal administration of exosomes after SE maintains normal hippocampal neurogenesis.

FIG. 8A-FIG. 8E—A1-exosomes invade the fronto-parietal cerebral cortex and the dorsal hippocampus within 6 hours after IN administration. FIGS. 8A1-8C2 show the presence of PKH26+ exosomes (red dots) within the cytoplasm or in close contact with the cell membrane of neuron-specific nuclear antigen positive (NeuN+) neurons in the cerebral cortex (8A1), the dentate hilus and granule cell layer (8B1) and CA3 pyramidal neurons (8C1) of the hippocampus at 6 hours after their IN administration. 8A2, 8B2 and 8C2 show magnified views of boxed regions in 8A1, 8B1 and 8C1. FIG. 8D shows lack of exosomes within the soma of glial fibrillary acidic protein positive (GFAP+) astrocytes and the presence of some exosomes adjacent to astrocyte processes. FIG. 8E demonstrates the presence of exosomes within the soma or processes of some IBA-1+ microglia. CA3-SP, CA3 stratum pyramidale; CA3-SR, CA3 stratum radiatum; CTX, cortex; DH, dentate hilus; GCL, granule cell layer. Scale bar, 8A1, 8B1, 8C1=50 μm; 8A2, 8B2, 8C2=25 μm 8D, 8E=25 μm.

FIG. 9A-FIG. 9J—Intranasal (IN) administration of A1-exosomes two hours after SE reduces the loss of neuron-specific nuclear antigen positive (NeuN+) neurons and parvalbumin positive (PV+) interneurons in the dentate gyrus and the CA1 subfield, when examined 4 days post-SE. FIG. 9A1-9C3 illustrate the distribution of NeuN+ neurons in the dentate gyrus (FIG. 9A1, 9B1, 9C1), the CA1 subfield (9A2, 9B2, 9C2) and the CA3 subfield (9A3, 9B3, 9C3) of a naïve control mouse (FIG. 9A1-9A3), a mouse that received vehicle after SE (SE-VEH group, 9B1-9B3) and a mouse that received A1-exosomes after SE (SE-EVs group, 9C1-9C3). Bar charts in FIGS. 9D-9E compare the numbers of NeuN+ neurons in the DH (FIG. 9D) and the CA1 pyramidal cell layer (FIG. 9E) of the hippocampus. While both SE groups display reduced numbers of NeuN+ neurons in comparison to the naïve control group, the SE-EVs group exhibits greater numbers of surviving neurons than the SE-VEH group, implying neuroprotection after IN administration of A1-exosomes. FIGS. 9F1-9H3 illustrate the distribution of PV+ interneurons in the dentate gyrus (9F1, 9G1, 9H1), the CA1 subfield (9F2, 9G2, 9H2) and the CA3 subfield (9F3, 9G3, 9H3) of a naïve control mouse (9F1-9F3), a mouse from the SE-VEH group (9G1-9G3) and a mouse from the SE-EVs group (9H1-9H3).

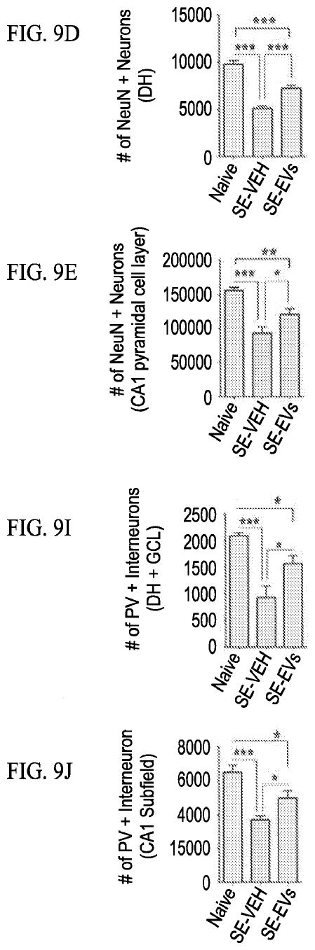

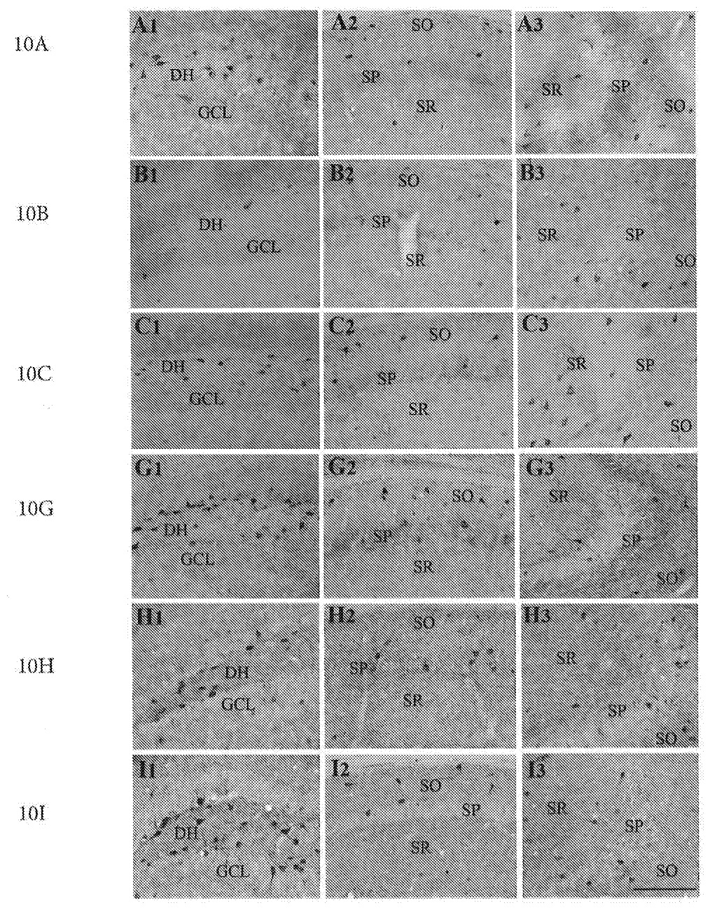

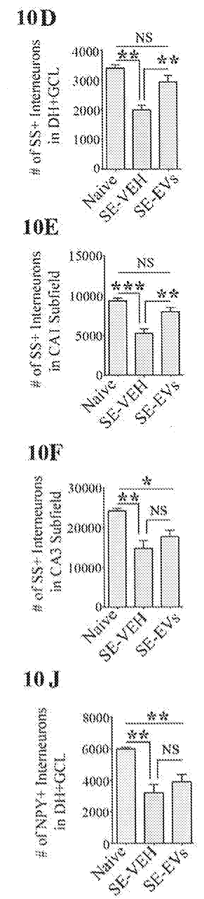

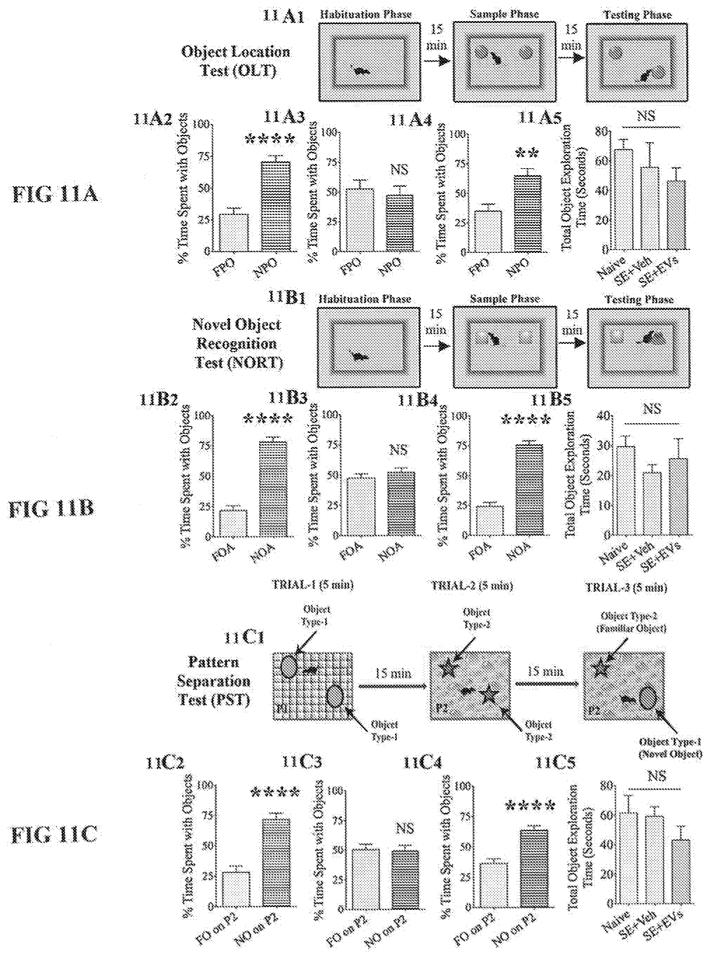

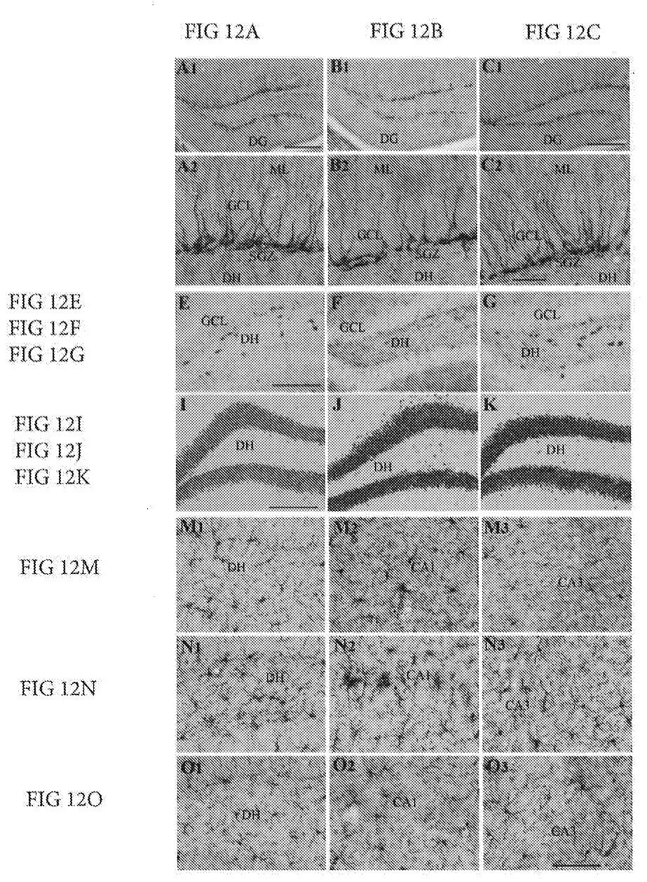

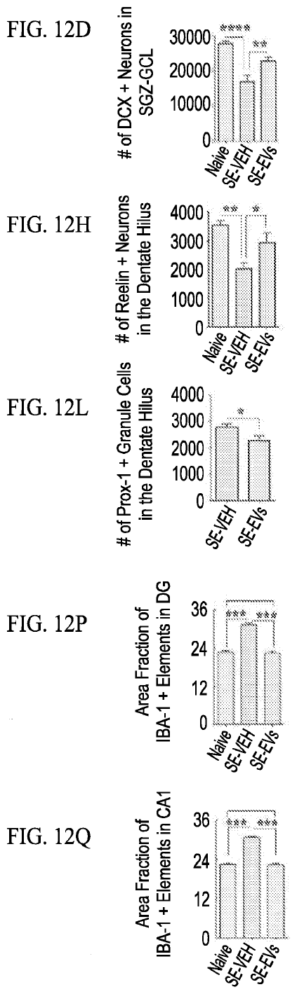

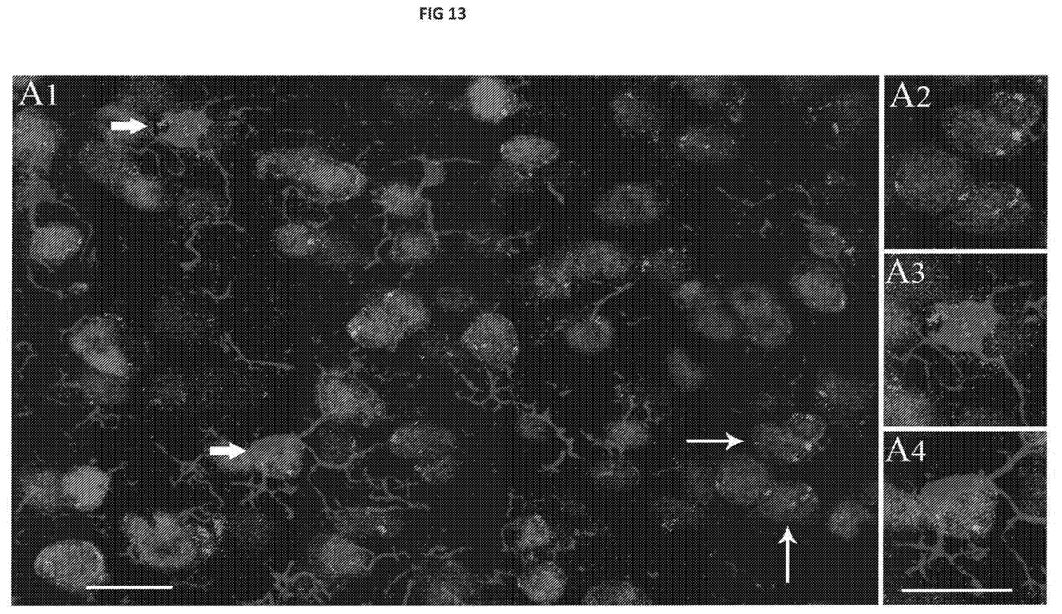

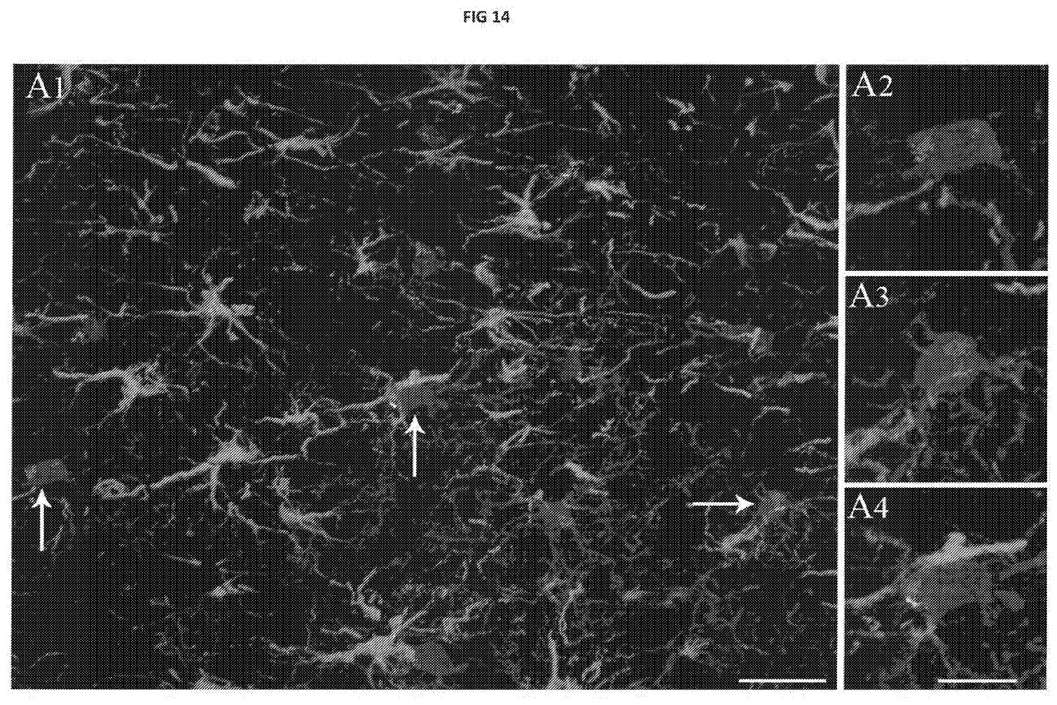

Bar charts in FIG. 9I-9J compare the numbers of PV+ interneurons in the dentate hilus and the granule cell layer (DH+GCL, I) and the CA1 subfield (J) of the hippocampus. While both SE groups display reduced numbers of PV+ interneurons in the DH+GCL and CA1 subfield in comparison to the naïve control group, the SE-EVs group exhibits greater numbers of PV+ interneurons than the SE-VEH group, implying protection of these interneurons after IN administration of A1-exosomes. DH, dentate hilus; GCL, granule cell layer; SO, stratum oriens, SP, stratum pyramidale; SR, stratum radiatum. Scale bar, 200 μm.*, p<0.05; **, p<0.01; ***, p<0.001.

FIG. 10A-FIG. 10J—Intranasal (IN) administration of A1-exosomes two hours after SE reduces the loss of somatostatin positive (SS+) and neuropeptide Y+ (NPY+) interneurons in the hippocampus, when examined 4 days post-SE. Panels 10A1-10C3 illustrate the distribution of SS+ interneurons in the dentate gyrus (10A1, 10B1, 10C3), the CA1 subfield (10A2, 10B2, 10C2) and the CA3 subfield (10A3, 10B3, 10C3) of a naïve control mouse (10A1-10A3), a mouse that received vehicle after SE (SE-VEH group, 10B1-10B3) and a mouse that received A1-exosomes after SE (SE-EVs group, 10C1-10C3). Bar charts in FIG. 10D-FIG. 10 F compare the numbers of SS+ interneurons in the dentate hilus+granule cell layer (DH+GCL; 10D) and the CA1 and CA3 subfields (10E, 10F) of the hippocampus. All regions display a significant loss of SS+ interneurons in the SE-VEH group but only the CA3 subfield shows some loss in the SE-EVs group. Overall, the SE-EVs group exhibits greater numbers of SS+ interneurons than the SE-VEH group in all regions, implying a considerable protection after IN administration of A1-exosomes. FIGS. 10G1-10I3 illustrate the distribution of neuropeptide Y+ (NPY+) interneurons in the dentate gyrus (10G1, 10H1, 10I1), the CA1 subfield (10G2, 10H2, 10I2) and the CA3 subfield (10G3, 10H3, 10I3) of a naïve control mouse (10G1-10G3), a mouse from the SE-VEH group (10H1-10H3) and a mouse from the SE-EVs group (10I1-10I3). Bar chart in FIG. 10J compares the numbers of NPY+ interneurons in the DH+GCL (10I) of the hippocampus. While both SE groups display reduced numbers of NPY+ interneurons in the DH+GCL in comparison to the naïve control group, the SE-EVs group exhibits relatively greater numbers of PV+ interneurons than the SE-VEH group, implying some protection of these interneurons after IN administration of A1-exosomes. DH, dentate hilus; GCL, granule cell layer; SO, stratum oriens, SP, stratum pyramidale; SR, stratum radiatum. Scale bar, 200 μm. *, p<0.05; **, p<0.01; ***, p<0.001.

FIG. 11A-FIG. 11C—Intranasal administration of A1-exosomes after SE prevents cognitive, memory and pattern separation impairments. FIGS. 11A1, 11B1 and 11C1 graphically depict the various phases involved in an object location test (OLT, 11A1), a novel object recognition test (NORT, 11B1), and a pattern separation test (PST, 11C1). Bar charts in FIG. 11A2-FIG. 11A4, FIG. 11B2-FIG. 11B4 and FIG. 11C2-11C4 compare percentages of time spent with different objects. Naive control animals showed a greater affinity for: (i) the novel place object (NPO) over the familiar place object (FPO) in an OLT (11A2); (ii) the novel object area (NOA) over the familiar object area (FOA) in an NORT (B2); and (iii) novel object on pattern 2 (NO on P2) over the familiar object on pattern 2 (FO on P2) in an PST (C2), implying normal cognitive, memory and pattern separation function. However, animals receiving vehicle after SE (SE+Veh) were impaired in all three tests (11A3, 11B3, 11C3). This was evinced by their behavior of spending nearly equal amounts of the object exploration time with the FPO and NPO in OLT (FIG. 11A3), FOA and NOA in NORT (FIG. 11B3), FO on P2 and NO on P2 in PST (FIG. 11C3). In contrast, animals receiving A1-exosomes after SE (SE-EVs) showed a greater affinity for exploring the NPO in OLT (FIG. 11A4), NOA in NORT (FIG. 11B4), and NO on P2 in PST (FIG. 11C4), suggesting a similar cognitive, memory and pattern separation function as naïve control animals. The bar charts in FIG. 11A5, FIG. 11B5 and FIG. 11C5 show that animals in different groups explored objects for comparable durations. **, p<0.01, ****, p<0.0001.

FIG. 12 A-FIG. 12 Q—Intranasal administration of A1-exosomes two hours after SE restrains multiple adverse changes that are typically seen in the chronic phase after SE. In comparison to naïve control animals (FIG. 12A1-12A2, FIG. 12E, 12I, 12M1-12M3), animals receiving vehicle after SE (SE-VEH group) showed waning of hippocampal neurogenesis (FIG. 12B1-FIG. 12B2, doublecortin [DCX] immunostaining), loss of reelin+ interneurons in the dentate gyrus (FIG. 12F), aberrant migration of newly born prox-1+ granule cells into the dentate hilus (FIG. 12J), and persistent hippocampal inflammation (with increased density and hypertrophy of IBA-1+ microglia, FIG. 12N1-N3). In animals receiving A1-exosomes after SE (SE-EVs group), the extent of neurogenesis (FIG. 12C1-FIG. 12C2), the survival of reelin+ interneurons (G), and the morphology and density of IBA-1+ microglia (FIG. 12O1-IG 12O3) were comparable to that observed in naïve control animals (FIG. 12A1-FIG. 12A2, FIG. 12E, FIG. 12 I, FIG. 12M1-FIG. 12M3). In addition, aberrant migration of newly born cells into the dentate hilus was reduced (FIG. 12K) in these animals. DG, dentate gyrus; GCL, granule cell layer; ML, molecular layer; SGZ, subgranular zone; Bar charts compare numbers of DCX+ newly born neurons in the subgranular zone-granule cell layer (SGZ-GCL, 12D), reelin+ interneurons in the dentate hilus (12H), numbers of prox-1+ newly born granule cells in the dentate hilus (12L), and IBA-1+ microglia in the dentate gyrus (12P) and the CA1 subfield (12Q) between different groups. Note that, the extent of neurogenesis (12D), numbers of reelin+ interneurons (12H) and IBA-1+ structures (12P-12Q) in SE-EVs group animals were comparable to that seen in naïve control animals. In addition, SE-EVs animals showed reduced numbers of prox-1+ cells in the dentate hilus (12L), implying a reduced abnormal migration of newly born granule cells with A1 exosome treatment after SE. Scale bar, 12A1, 12B1, 12C1=200 μm; 12A2, 12B2, 12C2=50 μm; 12E-12G and 12I-12K=200 μm; M1-O3=100 μm.*, p<0.05; **, p<0.01; ***, p<0.001; ****, p<0.0001.

FIG. 13—A1-exosomes displayed comparable affinity towards neurons and microglia. The panel A1 illustrates the distribution of A1-exosomes within NeuN expressing neurons and IBA-1 positive microglia in the anterior most part of the motor cortex at 6 hours after intranasal administration. Note that A1-exosomes are seen in the cytoplasm of majority of neurons in this region though the density of exosomes varied between neurons. The panel A2 shows a magnified view of neurons from panel A1 (indicated by thin arrows) displaying clumps of exosomes. Panel A1-exosomes also incorporated into the cytoplasm of all microglia in this region (panel A1). The panels A3 and A4 illustrate magnified views of microglia from panel A1 (indicated by thick arrows). One of these microglia displays clusters of exosomes in the soma (panel A3) while the other shows scattered exosomes in the soma and processes (panel A3). Scale bar, A1-A5, 20 μm.

FIG. 14—A1-exosomes showed greater affinity for microglia in comparison to astrocytes. The panel A1 illustrates GFAP positive astrocytes (green), IBA-1 positive microglia (blue) and panel A1-exosomes (red) in the frontal association cortex at 6 hours after intranasal administration. Note that clusters of A1-exosomes are seen in the cytoplasm of virtually all microglia in this region. The panels A2-A4 show magnified views of microglia from panel A1 (indicated by arrows) displaying larger clumps of exosomes. Interestingly, exosomes are not found in the soma of astrocytes but scattered exosomes are seen in close proximity to processes of astrocytes (panel A1). Scale bar, A1, 25 μm; A2-A4, 10 μm.

### DETAILED DESCRIPTION OF THE PREFERRED EMBODIMENTS

Throughout the specification and claims, the following terms take the meanings explicitly associated herein, unless the context clearly dictates otherwise.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

As used herein, the term “or” is an inclusive “or” operator and is equivalent to the term “and/or” unless the context clearly dictates otherwise.

The term “based on” is not exclusive and allows for being based on additional factors not described, unless the context clearly dictates otherwise.

The term “a,” “an,” and “the” include plural references. Thus, “a” or “an” or “the” can mean one or more than one. For example, “a” cell and/or extracellular vesicle can mean one cell and/or extracellular vesicle or a plurality of cells and/or extracellular vesicles.

The meaning of “in” includes “in” and “on.”

As used herein, “stem cell” refers to a multipotent cell with the potential to differentiate into a variety of other cell types (which perform one or more specific functions), and have the ability to self-renew.

As used herein, “adult stem cells” refer to stem cells that are not embryonic stem cells. By way of example, the adult stem cells include mesenchymal stem cells, also referred to as mesenchymal stromal cells or MSCs.

As used herein, the terms “administering”, “introducing”, “delivering”, “placement” and “transplanting” are used interchangeably and refer to the placement of the extracellular vesicles of the technology into a subject by a method or route that results in at least partial localization of the cells and/or extracellular vesicles at a desired site. The cells and/or extracellular vesicles can be administered by any appropriate route that results in delivery to a desired location in the subject where at least a portion of the cells and/or extracellular vesicles retain their therapeutic capabilities. By way of example, a method of administration includes intravenous administration (i.v.).

As used herein, the term “treating” includes reducing or alleviating at least one adverse effect or symptom of a disease or disorder through introducing in any way a therapeutic composition of the present technology into or onto the body of a subject.

As used herein, “therapeutically effective dose” refers to an amount of a therapeutic agent (e.g., sufficient to bring about a beneficial or desired clinical effect). A dose could be administered in one or multiple administrations (e.g., 2, 3, 4, etc.). However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., cells and/or extracellular vesicles as a pharmaceutically acceptable preparation) for aggressive vs. conventional treatment.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “pharmaceutical preparation” refers to a combination of the A1 exosomes, with, as desired, a carrier, inert or active, making the composition especially suitable for diagnostic or therapeutic use in vitro, in vivo, or ex vivo.

As used herein, the terms “pharmaceutically acceptable” or “pharmacologically acceptable” refer to compositions that do not substantially produce adverse reactions, e.g., toxic, allergic, or immunological reactions, when administered to a subject. For example, normal saline is a pharmaceutically acceptable carrier solution.

As used herein, the terms “host”, “patient”, or “subject” refer to organisms to be treated by the preparations and/or methods of the present technology or to be subject to various tests provided by the technology.

The term “subject” includes animals, preferably mammals, including humans. In some embodiments, the subject is a primate. In other preferred embodiments, the subject is a human.

The following examples are provided to demonstrate and further illustrate certain preferred embodiments and aspects of the present technology, and they are not to be construed as limiting the scope of the technology.

### Example 1—Method of Preparing the AI Exosome Intranasal Composition, the SE Animal Model

The procedure described in Kim et al (2016) was employed in the preparation of the A1 exosome preparation. Generally, a population of stem cells, such as mesenchymal stem cells, will be cultured under the conditions defined below, and the cell culture media in which the stem cells were cultured will be collected and screened to select a population of extracellular vesicles (EVs) having a defined set of characteristics.

Preparations of tissue-derived mesenchymal stem cells (MSCs) may vary in their characteristics depending on many factors, including the properties of the donor of the tissue and the tissue site from which the cells are obtained from the same donor. A preparation of MSCs derived from human bone marrow (defined as Donor 6015) from an NIH-sponsored center for distribution of MSCs was used that met the classical in vitro criteria for MSCs, and ranked among the top three of 13 MSC preparations in expression of the biomarker of mRNA for TSG-6 and in modulating inflammation in three murine models. Cell culture media collected from the culture of these MSCs was collected, and a population of EVs having a set of defined characteristics were selected and tested. One of the primary characteristics of the selected population of EVs was the ability to suppressed cytokine production.

To reduce variability and improve consistency in EV production, a protocol was followed in which the MSCs were consistently plated at 500 cells/cm² in a standardized medium containing 17% of a pre-tested batch of fetal bovine serum (defined as complete culture medium or CCM). The CCM was replaced after 2 or 3 days.

After 5 days, the cell culture medium was changed to a chemically defined and protein free medium (CDPF) that had been optimized for production of recombinant proteins by Chinese hamster ovary cells (Invitrogen). The medium was further supplemented with the components of Table 1 to minimize aggregation of cells secreting TSG-6. Aggregation is caused by cross-linking of hyaluronan on the cell surface.

TABLE 1
CDPF media for the preparation of hMSC-derived
extracellular vesicles (exosomes)

| | Concen- | | |
|---|---|---|---|
| Components | trations(/L) | | Sources |
| CD-CHO protein-free | 925 | ml | Invitrogen: 107 43-011 |
| medium | | | |
| HT supplements* | 10 | ml | Invitrogen: 11 067 -030 |
| 200 mM L-glutamine | 40 | ml | lnvitrogen: 25030-081 |
| D-[+]-glucose | 2 | g | Sigma: G6152-100g |
| 100x Non-essential | 10 | ml | lnvitrogen: 11140-050 |
| amino acid | | | |
| 100x MEM vitamin | 10 | ml | lnvitrogen: 11120-052 |
| solution | | | |

*A mixture of hypoxanthine (10 mM) and thymidine (1.6 mM).

As a convenient marker for the EVs, assays for CD63, a tetraspan protein in EVs, was used. Culture of MSCs in the CDPF medium was found to increase the expression of mRNA for CD63. The expression of the mRNA for CD63 increased for at least 48 hours and was accompanied by the accumulation of the CD63 protein in the medium. However, the pattern of genes expressed differed during the time of incubation in the CDPF. At 2 hours, there was a high level of expression of mRNA for IL-10, a major pro-inflammatory cytokine. In contrast, expression of mRNA for the inflammation modulating protein TSG-6 was low at 2 hours and increased progressively at 6, 24 and 48 hours. The TSG-6 protein in medium did not increase until about 48 hours. On the basis of these observations, a standardized protocol for production on EVs having anti-inflammatory properties was developed.

The MSCs did not expand but there was little evidence of cell death during their incubation in the CDPF medium for 48 hours. Comparison of preparations of MSCs demonstrated that the levels of CD63 protein in the harvested medium were higher in the preparation from MSCs of Donor 6015, compared to three other preparations. Also, the level of TSG-6 in the harvested medium was found to be the highest in culture medium collected from MSC cells obtained from Donor 6015.

Isolation of EVs with a Scalable Protocol. Most of the published protocols for isolation of EVs involve high speed centrifugation or other procedures that cannot be readily scaled up for large scale production. To develop a scalable protocol, EVs from the harvested medium by chromatography was developed. Most of the protein in the harvested medium was found to be bound to an anion exchange resin, but little of the protein in the medium bound to a cation exchange resin. A protocol was developed in which the harvested medium was centrifuged at 2,500×g for 15 min and then the supernatant was collected and chromatographed on an anion exchange column. The protein that eluted with 0.5 M NaCl was recovered as a single broad peak, and the protein contained CD63. The recovery of CD63 in the peak ranged from 73% to 81% (n=3), and was slightly higher than was obtained by centrifuging the harvested medium at 100,000×g for 12 hours. Assay of the peak fractions with a nanoparticle tracking system demonstrated that they contained about 0.51×10⁹ vesicles per μg protein. Assays at decreasing concentrations indicated that the mean size of the vesicles was 231+/−3.2 nm (SEM), 216+/−2.3 nm, and 207+/−1.8 nm. Of interest was that the three peaks observed at the lowest concentration were 85, 165 and 236 nm, the expected sizes of EVs of 85 nm that were also recovered as dimers and trimers.

Surface Epitopes of the Isolated EVs. To map surface epitopes of the EVs, the method of Oksvoid et al. (Methods Mol. Biol., 1218:465-481) was used, whereby EVs are first trapped with a large bead linked to an antibody to CD63, and then additional epitopes on the trapped EVs are assayed with standard protocols for flow cytometry. The EVs captured with the protocol were positive for CD63. They were also about 80% positive for CD81, another epitope frequently found on EVs. However, they were negative for CD9 a third epitope frequently found on EVs. Also, they were also negative for 13 epitopes found on the surface of MSCs (Table 2). Therefore the EVs probably arose from multivesicular bodies in the cytoplasm that are frequently referred to as exosomes.

TABLE 2
Surface Epitopes in hMSC's and EVs

| | hMSCs* | | |
|---|---|---|---|
| Surface epitopes | CCM | CDPF | EVs* |
| hMSC markers | | | |
| CD29 | >99 | >99 | <1 |
| CD44 | >99 | >99 | <2 |
| CD49c | >99 | >99 | <1 |
| CD49f | >99 | >99 | <1 |
| CD59 | >99 | >99 | 2.04 |
| CD73 | >99 | >99 | <2 |
| CD90 | >99 | >99 | <1 |
| CD105 | >99 | >99 | <1 |
| CD146 | >99 | >99 | <2 |
| CD147 | >99 | >99 | <1 |
| CD166 | >99 | >99 | <1 |
| HLA-a, b, c | >99 | >99 | <2 |
| PODXL | 95 | 91 | <2 |
| EV markers | | | |
| CD9 | 93 | 99 | <1 |
| CD63 | 48 | 85 | 90.6 |
| CD81 | >99 | >99 | 79.9 |

hMSC, human mesenchymal stem/stromal cell.
*Positively stained cells or EVs (% of total) with antibodies indicated in Table S2.

Culture Conditions for Producing EVs—A frozen vial of passage 4 hMSCs from bone marrow <(medicine.tamhc.edu/irm/mscdistribution.html)> was thawed at 37° C., and plated directly at around 500 cells cm² in 150×20 mm-diameter tissue culture plates in 30 mL of complete culture medium (CCM) that consisted of α-MEM, 16.6% FBS, 100 unites/mL penicillin, 100 μg/ml streptomycin and 2 mM L-glutamine. The CCM medium was replaced after 2-3 days. After the cells reached about 70% confluency in about 4-6 days, the medium was replaced with a medium optimized for Chinese Hamster ovary cells (CD-CHO medium; cat no 10743-002, Invitrogen), that was further supplemented to prevent aggregation of cells synthesizing TSG-6 (See Table 1). The medium was recovered after 6 hours, to be assayed, and discarded. The medium was replaced and the medium was recovered between 6 and 48 hours was either stored at −80° C. or used directly to isolate EVs.

The CDFM medium collected after 48 hours was centrifuged at 2,465×g, 15 minutes, to remove any cells and debris. This media was centrifuged again at 100,000×g (Sorvall WX Floor Ultra Centrifuge and AH-629 36 mL swinging Bucket Rotor; Thermo) for 1, 5, and 12 hours at 4° C. EVs were stored in PBS at 4° C. or −20° C. EV protein content was quantified by the Bradford method (Bio-Rad).

The EVs were isolated by chromatography, by applying the supernatant to an anion exchange column, and the column was eluted with 500 mM NaCL. The protein eluted as a single broad peak that contained CD63. The negatively charged extracellular vesicles (n-EVs) present in the broad peak eluted fractions were obtained.

The enriched populations of n-EVs, i.e., the A1 exosomes, may be distinguished from other vesicle preparations by reference to several characteristics. For example, the detectable surface epitopic characteristics of the preparations may be described as CD63+ and CD81+, and/or as being essentially absent detectable surface levels of (i.e., are negative for) CD9, and are essentially absent, or are identified to have less than 1%, less than about 2%, positively stained cells with antibodies to, any combination of two (2) or more, or all, of the surface epitopes CD29, CD44, CD49c, CD49f, CD59, CD73, CD90, CD105, CD146, CD147, CD166, HLA-a, b, c, and PODXL. Note these epitopes CD9, CD29, CD44, CD49c, CD49f, CD59, CD73, CD90, CD105, CD146, CD147, CD166, HLA-a, b, c, and PODXL, are present on the surface of the mesenchymal stem cells cultured to produce the population of extracellular vesicles that are ultimately formulated in the preparations of n-EVs of the present invention.

In addition, the n-EV preparations of the present invention, the A1 exosomes, as well as the compositions that contain them, are suitable for use in humans. They may be formulated as part of an injectable preparation or as a formulation suitable for intranasal administration, so as to provide a pharmaceutically acceptable preparation. As part of an injectable preparation, the n-EVs may be formulated in a pharmaceutically acceptable carrier solution, such as saline. In a formulation suitable for intranasal administration, the EVs may be formulated in phosphate buffered saline (PBS), a buffer solution that is a water-based salt solution containing disodium hydrogen phosphate, sodium chloride, and in some formulations, potassium chloride and potassium dihydrogen phosphate.

Alternatively, the n-EVs may be contained in a biologically compatible drug delivery depot, such as a depot that may be surgically implanted into a patient. The depot would permit the n-EVs to be delivered into the system of the patient, thus providing the intended therapeutic effect.

The n-EV preparations may also be described as a human n-EV preparation, as they are prepared from human mesenchymal stem cells, obtained from a human tissue source, such as bone marrow.

The A1 exosomes were then prepared to provide an EV A1 formulation having a final concentration of about 200 μg protein/mL. in a sterile saline solution. This formulation was stored at −80° C. until administration. The A1 exosome preparation may be described as comprising about 15×10⁹ EVs. The preparations may also comprise a pharmaceutical dose of A1 exosomes that includes about 20 μg protein to about 30 μg protein. Only about 4 ng of native TSG-6 protein was identified in the A1 exosome preparation. Prior reports describe the use of preparations that included 50 μg of recombinant TSG-6 for inflammation in four models on induced inflammation in mice.

SE Animal Model: In the present studies, an acute epilepsy model was employed. The acute seizure model used permits examination of conditions associated with neurodegeneration and severe inflammation in the hippocampus. These events physiologically evolve into cognitive and memory impairments as well as chronic epilepsy.

Intraperitoneal injection of pilocarpine (290-340 mg/Kg) in a mouse was performed to induce SE typified by continuous seizures.

To limit the duration of SE and mortality of animals, seizures were terminated with a diazepam injection (10 mg/kg) at 2 hours after the onset of SE. The severity of convulsive responses was monitored and classified according to the modified Racine scale.

Mice that displayed intermittent or continuous stage 4 seizures (bilateral forelimb myoclonus and rearing) or stage 5 seizures (bilateral fore- and hind-limb myoclonus and transient falling) were assigned randomly to the exosome receiving group or the vehicle-receiving group.

Study Design: For intranasal administration of exosomes, following termination of seizures using a diazepam injection, each nostril was treated with 5 μL of hyaluronidase (100 U, Sigma-Aldrich) in sterile PBS to increase the permeability of the nasal mucosa injection. Thirty minutes later, a suspension of exosomes or vehicle was slowly administered bilaterally into the nostrils using a small pipette (10 μL micropipette). The solution was ejected in 5-microliter increments every 10 minutes until 75-μL was administered to each mouse. A day later, additional 75 μL of exosomes or vehicle was administered to each mouse. Thus, each mouse received a total of 150 μL PBS or exosomes (30 μg, ˜15×10⁹ exosomes) over two days, commencing ˜2.5 hours after the induction of SE.

Selection of the A1 extracellular Vesicles—Assays for Anti-Inflammatory Activity of EVs: IL-6, IFN-γ, and IL-1β. Assays of Anti-Inflammatory Activity of EVs. C57BL/6 male mice (Jackson Laboratories) 6 to 8 weeks old were injected through a tail vein with 150 μl of PBS, 50 μg LPS from Escherichia coli 055:B5 (Sigma, L2880) in PBS, 50 μg LPS+30 μg Dexamethasone (Sigma, D4902) in PBS, or 50 μg LPS+EVs (30 μg protein and 15 billion vesicles) in PBS. After 3 hours, the mice were killed and the spleens assayed by RT-PCR with commercial kits for IL-6, IFN-γ, and IL-1β using β-actin as an internal standard. EVs that did not produce a significant decrease (p<0.05) in all three of the pro-inflammatory factors were rejected for further use. Batches of EVs that decreased the levels of all three pro-inflammatory cytokines were chosen and referred to as A1-exosomes.

Labeling of A1-exosomes. A1-exosomes were labeled with the red fluorescent membrane dye PKH26 (Sigma, MINI26). This was done by transferring A1-exosomes from PBS to diluent C solution (Sigma) by centrifugation at 100,000×g for 70 min. PKH26, diluted to 4 mM, and the A1-exosomes (200 μg/ml) were filtered separately through small 0.2 μm syringe filters before mixing at 1:1 for 5 min, followed by the addition of 5% BSA and washing by centrifugation. The pellet of A1-exosomes was suspended in 0.5 ml PBS. To avoid dye-stained aggregates, the A1-exosomes were filtered through a 0.2 μm syringe filter immediately before use.

Animals. Male C57BL/6J mice were purchased from the Jackson Laboratory. They were 6-8 weeks old at the time of commencement of experiments. Animals were housed in an environmentally controlled room with a 12:12-hr light-dark cycle and were given food and water ad libitum. All animals were treated in accordance with a protocol approved by the Institutional Animal Care and Use Committee of Texas A&M Health Science Center College of Medicine.

Induction of Status Epilepticus (SE). Animals first received a subcutaneous (SQ) injection of scopolamine methyl nitrate (1 mg/kg, Sigma-Aldrich, S2250), as a measure to reduce the peripheral cholinergic effects of pilocarpine. Following a waiting period of 30 minutes, animals received an intraperitoneal injection of pilocarpine hydrochloride (Sigma-Aldrich, P6503) at a dose of 290-350 mg/Kg (59-61), which induced SE. Animals were closely monitored for the severity and length of the behavioral seizures. To limit the duration of SE and mortality to comparable periods, seizures were terminated in all animals with a diazepam injection (10 mg/kg, SQ.) at 2 hours after the onset of SE. The severity of convulsive responses was monitored and classified according to the modified Racine scale (62). Mice that showed consistent stage 4 (i.e. bilateral forelimb myoclonus and rearing) or stage 5 (i.e. bilateral fore- and hind-limb myoclonus and transient falling) seizures were chosen for further experimentation. Animals that did not show consistent acute seizure activity (i.e. non-responders exhibiting either no seizures or isolated milder seizures) were excluded from the study. Furthermore, animals that demonstrated extensive and severe tonic-clonic seizures (over-responders) were euthanized to avoid severe pain and distress.

Intranasal administration of A1-exosomes or vehicle. A1-exosomes were prepared using sterile PBS at a concentration of 200 μg/ml and stored at −80° C. Mice that displayed SE after a pilocarpine injection were randomly assigned to the vehicle (PBS administration, SE+Veh group) or A1-exosomes group (also referred to as SE+EVs group). Following termination of two hours of SE through a diazepam injection, each nostril was treated with 5 μl of hyaluronidase (100 U, Sigma-Aldrich, H3506) in sterile PBS to enhance the permeability of the nasal mucous membrane. Thirty minutes later, each mouse was held ventral-side up with the head facing downwards. Each nostril was then carefully administered with PBS or A1-exosomes in ˜5 μl spurts separated by 5 minutes interval, using a 10 μl micropipette. Each mouse received a total volume of 75 μl on SE day. Eighteen hours later, another 75 μl of PBS or A1-exosomes was administered in a similar manner. Overall, each mouse received a total of 150 μl of either PBS or A1 exosomes (30 μg, about 15×10⁹) within 18 hours after 2 hours of SE. However, mice in A1-exosome tracking studies received administration (75 μl) of A1-exosomes only on SE day.

Tracking of intranasally administered A-1 exosomes. For tracking intranasally administered exosomes, PKH26-labeled exosomes (15 μg, about 7.5 billion per mouse) were administered only on SE day, using methods described above (n=4). Each mouse was transcardially perfused with saline and paraformaldehyde (PH=7.4) at 6 hours following A1-exosome administration. The brains were removed, post-fixed in 4% paraformaldehyde overnight, and cryoprotected with different grades of sucrose solution. Thirty-micrometer thick coronal sections were cut through the entire brain using a cryostat and the sections were collected serially in 24-well plates containing phosphate buffer (PB). Representative sets of sections (every 10^{th}) through different levels of the cortex and the hippocampus were chosen for tracking the intranasally administered A1-exosomes through dual immunofluorescence and confocal microscopy. Briefly, different sets of sections were labelled with primary antibodies for NeuN (a pan neuronal marker, Millipore, ABN78), GFAP (a marker of astrocytes, Millipore, MAB360), or IBA-1 (a marker of microglia, Abcam, ab5076). The secondary antibodies comprised Cy2 conjugated donkey anti-goat IgG (Jackson Immuno Research, 715-225-150), Cy2 conjugated donkey anti-rabbit IgG (Jackson Immuno Research, 711-545-152) or A488 anti-mouse IgG (Thermo Fisher Scientific, A-21202). Sections were mounted using an antifade reagent (Sigma, S7114). One μm-thick optical Z-sections were sampled from different regions of the cortex and various subfields of the hippocampus using a confocal microscope (FV10i, Olympus or Ti-Eclipse, Nikon) and the images were analyzed using Olympus FV-10i image browser.

Preparation of hippocampal extract and evaluation of cytokines. Twenty four hours after the first administration of PBS or A1-exosomes (i.e. 75 μl administration performed ˜2 hours after SE induction), mice were anesthetized with isoflurane and the brains were rapidly dissected from the skull and stored at −80° C. (n=6/group). For comparison, brains were similarly collected from naïve control animals (n=6). For the evaluation of cytokine levels in the hippocampus, each brain was thawed, the hippocampus was rapidly dissected under a stereomicroscope and sonicated on ice in lysis buffer containing protease inhibitor cocktail (Sigma, P2714), and centrifuged at 10,000 RPM for 5 minutes at 4° C. The supernatant was collected, the total protein concentration was measured and the lysate was diluted for the required concentration. Each 96-well cytokine array plate (Signosis, EA-4005) used in this study displayed 4 segments (24 wells/segment) adequate for measuring 24 different cytokines from four samples. The wells were pre-coated with specific cytokine capture antibodies. The assay was performed as per the manufacturer's guidelines with each well receiving 10 μg of lysate (in 100 μl volume). In this assay, the concentration of each of 24 cytokines in hippocampal lysates is directly proportional to the intensity of color. For TNF-a (Signosis, EA-2203) and IL1-β (Signosis, EA-2508) enzyme-linked quantitative immunoassays, 100 μl of serially diluted standard and 100 μl hippocampal lysate were used. The assay was performed as per the manufacturer's guidelines. The levels of TNF a and IL1-β were quantified using the standard graph and expressed as pg/mg of protein.

Tissue processing and immunohistochemistry. Four days after SE, subgroups of animals belonging to both SE+Veh and SE+EVs group (n=6/group) were perfused with 4% paraformaldehyde, as described in the earlier section. A subgroup of age-matched naïve control animals (n=5) were also perfused similarly. Several sets of serial sections (every 15^{th} or 20^{th}) through the entire hippocampus were selected and processed for immunohistochemistry. The procedures employed for immunostaining are recognized by those of skill in the art. Briefly, the sections were etched with PBS solution containing 20% methanol and 3% hydrogen peroxide for 20 minutes, rinsed thrice in PBS, treated for 30 minutes in PBS containing 0.1% Triton-X 100 and an appropriate serum (10%) selected on the basis of the species in which the chosen secondary antibody was raised. The primary antibodies comprised anti-CD68 (ED-1, an activated microglia marker; Bio-Rad Laboratories, MACA341R), anti-NeuN (a pan neuronal marker; Millipore, ABN78), anti-parvalbumin (PV, a calcium binding protein found in a subclass of GABA-ergic interneurons; Sigma-Aldrich, P3088), anti-somatostatin (SS, a neuropeptide found in a subclass of GABA-ergic interneurons; Peninsula Laboratories, T-4546) or anti-neuropeptide Y (NPY, another neuropeptide found in a subclass of GABA-ergic interneurons; Peninsula Laboratories, T-4070). After an overnight incubation with the respective primary antibody solution, sections were washed thrice in PBS and incubated in an appropriate secondary antibody solution for an hour. Biotinylated anti-rabbit (H+L) (Vector Lab, BA-1000) and biotinylated anti-mouse (H+L) (Vector Lab, BA-2000) were used for the study. The sections were washed thrice in PBS and treated with avidin-biotin complex reagent (Vector Lab, PK-6100) for an hour. Peroxidase reaction was developed using diaminobenzidine (Vector Lab, SK-4100) or vector SG (Vector Lab, SK-4700) as chromogens, and the sections were mounted on gelatin coated slides, dehydrated, cleared and cover slipped with permount.

Measurement of the number of various immunostained cells. The optical fractionator method in the StereoInvestigator system (Microbrightfield Inc., Williston, Vt.) interfaced with a Nikon E600 microscope through a color digital video camera (Optronics Inc., Muskogee, Okla.) was employed for all cell counts performed at 4 days post-SE. This comprised quantification of numbers of: (i) activated microglia positive for ED-1 in the dentate gyrus (DG) and CA1 and CA3 subfields; (ii) neurons positive for NeuN in the dentate hilus (DH) and the CA1 pyramidal cell layer; (iii) interneurons positive for PV, SS and NPY in the DH+granule cell layer (GCL) and CA1 and CA3 subfields. A detailed methodology employed in these counts is described in previous reports (5,63).

Behavioral tests. Animals were examined with three object based tests at 5-6 weeks after SE in SE+Veh and SE+EVs groups (n=8-10/group). Age-matched naïve control animals (n=10) were also included for comparison. The tests comprised an object location test (OLT), a novel objection recognition test (NORT) and a pattern separation test (PST). All tests were performed using an open field apparatus (measuring 45×45 cm).

Object location test (OLT). Each mouse was observed in an open field with three successive trials separated by 15 minute intervals. A detailed description of this test is available in a previous report. In brief, the mouse was placed in an open field for 5 minutes in the first trial for acclimatization to the testing apparatus (habituation phase) whereas in trial 2, the mouse was allowed to explore two identical objects placed in distant areas of the open field (sample phase). In trial 3 (testing phase), one of the objects was moved to a new area (novel place object, NPO) while the other object remained in the previous place (familiar place object, FPO). Both trials 2 and 3 were video recorded using Noldus-Ethovision video-tracking system to measure the amount of time spent with each of the two objects. Exploration of the object was defined as the length of time a mouse's nose was 1 cm away from the marked object area. The results such as the percentage of object exploration time spent in exploring the NPO and FPO as well as the total object exploration time in trial 3 were computed. The percentage of time spent with the NPO and FPO was calculated by using the following formula: the time spent with the particular object/the total object exploration time×100.

Novel object recognition test (NORT). Each mouse was examined in an open field with three consecutive trials separated by 15 minute intervals. A detailed description of this test is available in a previous report (32). The first two trials comprised an acclimatization period of 5 minutes to an open field (trial 1 or habituation phase) and exploration of two similar objects placed in distant areas within the open field for 5 minutes (sampling phase). In trial 3 (testing phase), the mouse was allowed to explore a different pair of objects comprising one of the objects used in trial 2 and a novel object for 5 minutes. Both trials 2 and 3 were video recorded using Noldus-Ethovision video-tracking system. The amounts of time spent with the familiar object area (FOA) and the novel object area (NOA) and the total object exploration time in trial 3 were computed. Exploration of the FOA or NOA was defined as the length of time a mouse's nose was 1 cm away from the respective area. The percentages of time spent with the NOA or FOA were calculated by using the following formula the time spent with the particular object/the total object exploration time×100.

Analyses of hippocampal neurogenesis and inflammation in the chronic phase after SE. Animals were perfused 6 weeks post-SE using 4% paraformaldehyde. Tissue processing, section cutting and storage of sections were done as described earlier. Serial sections (every 10th) through the entire hippocampus were selected from animals belonging to naïve control, SE+Veh and SE+EVs groups (n=6/group) and processed for immunohistochemistry. The detailed procedures employed for immunostaining are available in a previous report (5). The primary antibodies comprised anti-doublecortin (anti-DCX, a marker of newly born neurons [63]; Santa Cruz Biotechnology, sc-8066; Abcam, ab5076), anti-reelin (marker of a subclass of interneurons that secrete reelin in the hippocampus; Millipore, MAB5364), Prox-1 (a marker of dentate granule cells; Millipore, AB5475) and anti-IBA-1 (a pan microglial marker; Abcam, ab5076). The secondary antibodies comprised biotinylated anti-goat (H+L) (Vector Lab, BA-9500), biotinylated anti-mouse (H+L) (Vector Lab, BA-2001), or biotinylated anti-rabbit (H+L) (Vector Lab, BA-1000). Following incubation in secondary antibody solutions, sections were incubated with avidin-biotin complex reagent (Vector Lab, PK-6100). The peroxidase reaction was developed using diaminobenzidine (Vector Lab, SK-4100) or vector SG (Vector Lab, SK-4700) as chromogens, and the sections were mounted on gelatin coated slides, dehydrated, cleared and cover slipped with permount.

Quantification of numbers of DCX+, prox-1+ and reelin+ neurons. An optical fractionator method available in the StereoInvestigator system (Microbrightfield Inc.) was employed for counting: (i) newly born neurons positive for doublecortin (DCX) in the subgranular zone-granule cell layer (SGZ-GCL) of the hippocampus; (ii) prox-1+ granule cells in the DH; and (iii) reelin+ interneurons in the DH. Quantification was done from 5-6 animals per group. A detailed methodology employed in these counts is described in a previous report (5, 63).

Measurement of the area fraction of IBA-1+ immunoreactive elements. The area occupied by IBA-1+ immunoreactive elements (soma and processes of microglia) in the DG and CA1 and CA3 subfields were quantified using Image J software, as described in a previous report (5). In brief, images from different regions of the hippocampus were digitized using a 20× objective lens in a Nikon E600 microscope equipped with a digital video camera connected to a computer. Each image saved in gray scale as a bitmap file was opened in Image J software, and a binary image was created through selecting a threshold value that retained all IBA-1+ structures but no background. The area occupied by the IBA-1+ structures (i.e. the area fraction) in the binary image was then measured by selecting the Analyze command in the program. Area fraction of IBA-1+ immunoreactive elements was calculated separately for every hippocampal region in each animal by using data from all chosen serial sections before the mean and SEM were determined for the total number of animals included per group (n=4/group).

Statistical analysis. Statistical analyses were performed using Prism software. One-way analyses of variance (one-way ANOVA) with Newman-Keuls multiple comparison post hoc tests was employed when 3 groups were compared. Comparison within groups in the behavioral tests or comparison between the two groups (e.g. prox-1 counts) employed unpaired, two-tailed Student's-t test. Numerical data were presented as mean±SEM and a p value less than 0.05 was considered as statistically significant.

The studies described in the following examples were conducted using the above A1 formulations, animal model and design study.

### Example 2—Intranasal Administration of A1 Exosomes (EVs) Target Hippocampus in an ES In Vivo Model

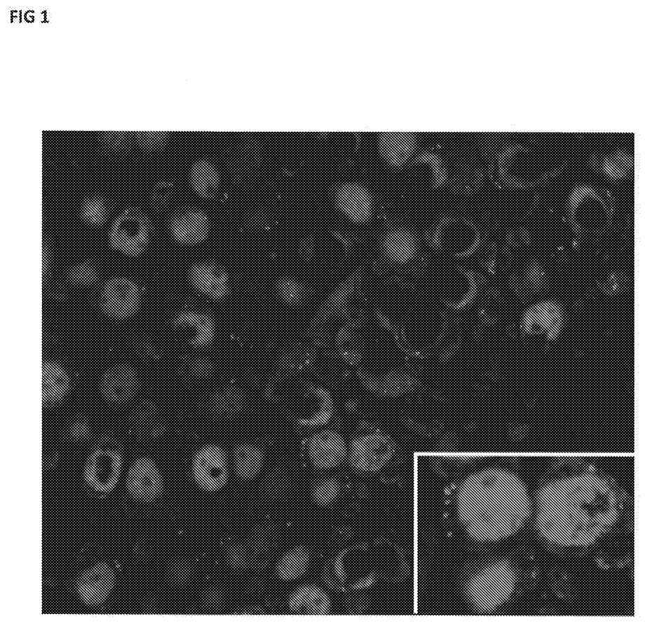

The present example demonstrates that in vivo intranasal administration of the A1 EV preparation reach the hippocampus (CA3 region) within 6 hours after intranasal administration. FIG. 1 shows distribution of PKH26 labeled EVs in the cytoplasm of CA3 pyramidal neurons of the hippocampus at 6 hours after intranasal administration.

Thus, intranasal administered EVs target the area of the hippocampus associated with neurodegeneration after SE.

### Example 3—Pro-Inflammatory Cytokine and Chemokine Levels in the Hippocampus In Vivo

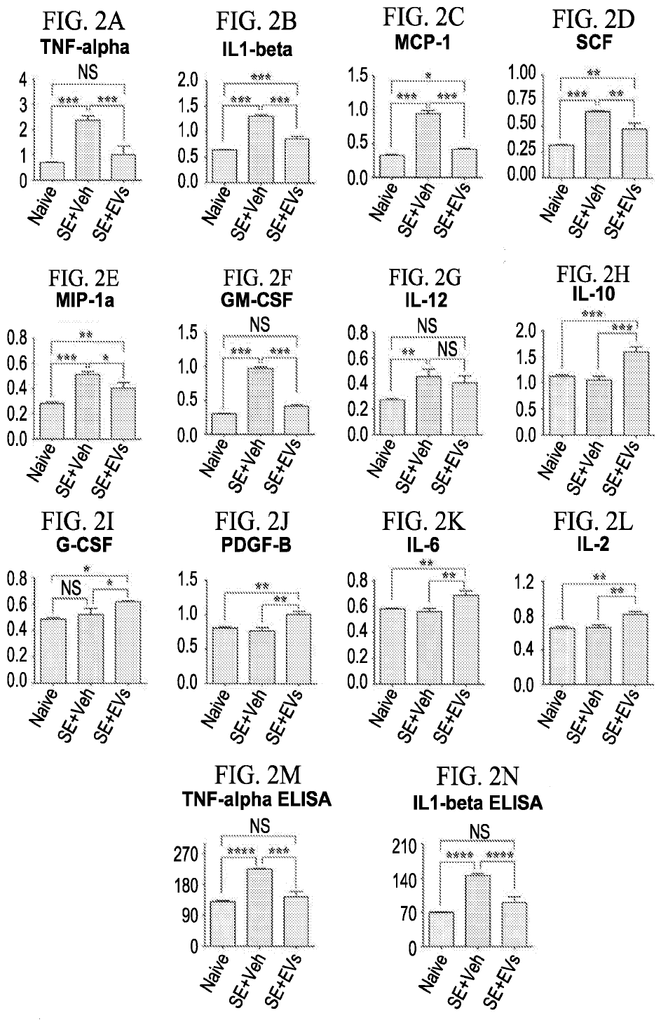

The results of this study are presented at FIG. 2.

This example demonstrates that intranasally administered EVs target a tissue, the hippocampus, which is the area of neurodegeneration after SE. This example also shows that this administration of EVs intranasally prevented the up-regulation of 9 different pro-inflammatory proteins (cytokines and chemokines in the hippocampus). These pro-inflammatory proteins include TNFa, IL-1b, MCP-1, MIP-1a, GMCSF, IL-12, SCF, IFNg and IGF-1.

EV administration intranasally is also shown here to enhance the concentration of anti-inflammatory cytokine IL-10 (see FIG. 2). Thus, intranasal EV treatment after SE significantly extinguishes a major inflammatory response in the hippocampus.

The above results demonstrate that EV treatment after SE significantly extinguishes a major inflammatory response in the hippocampus.

EV administration is also shown to enhance the concentration of anti-inflammatory cytokine IL-10. Thus, intranasal EV treatment after SE significantly extinguishes a major inflammatory response in the hippocampus.

### Example 4—A1 EVs and Glutamatergic Neurons in the Hippocampus

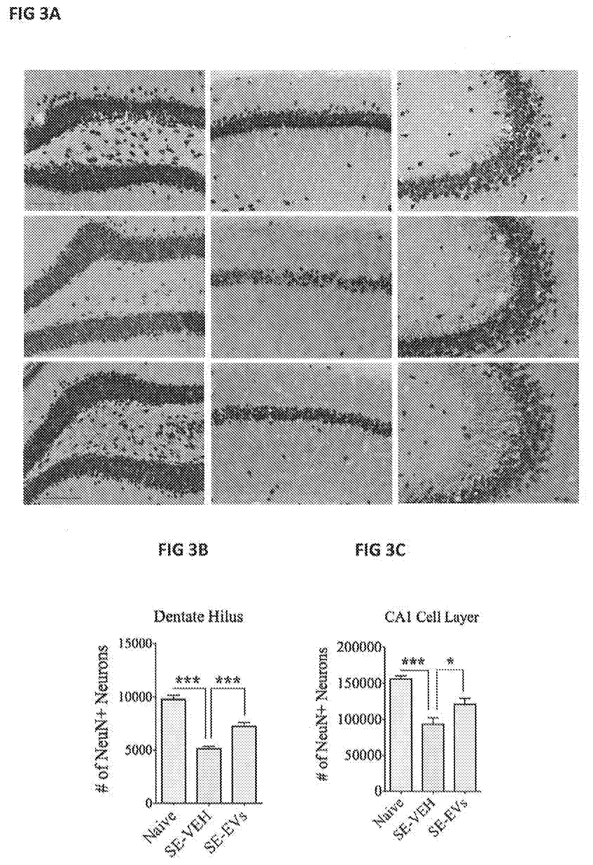

The results of this study are presented at FIG. 3.

Intranasal Administration of Exosomes after SE is shown to prevents the loss of glutamatergic neurons in the hippocampus: FIG. 3 shows a significant loss of neurons in the dentate hilus and the CA1 subfield of the hippocampus in a mouse that received vehicle after SE (FIG. 3, the first two photographs in the middle panel), in comparison to preservation of neurons in a mouse that received exosomes after SE (the first two figures in the bottom panel). Top panels of FIG. 3 provide data from a naïve control mouse.

### Example 5—A1 EVs and GABA-Ergic Interneurons in the Hippocampus

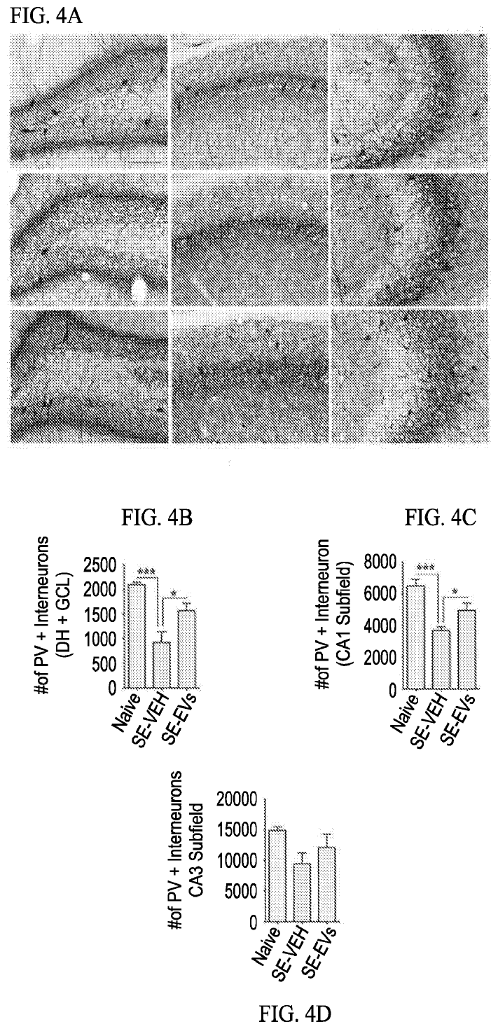

The results of this study are presented at FIG. 4.

FIG. 4 shows a significant loss of parvalbumin-positive GABA-ergic interneurons in the dentate gyrus and the CA1 subfield of the hippocampus in a mouse that received vehicle after SE (the first two photographs in the middle panel). In contrast, mice receiving EVs after SE showed preservation of neurons (the first two figures in the bottom panel). Top panels: examples from a naïve control mouse.

### Example 6—A1 EVs and Inflammation in the Hippocampus

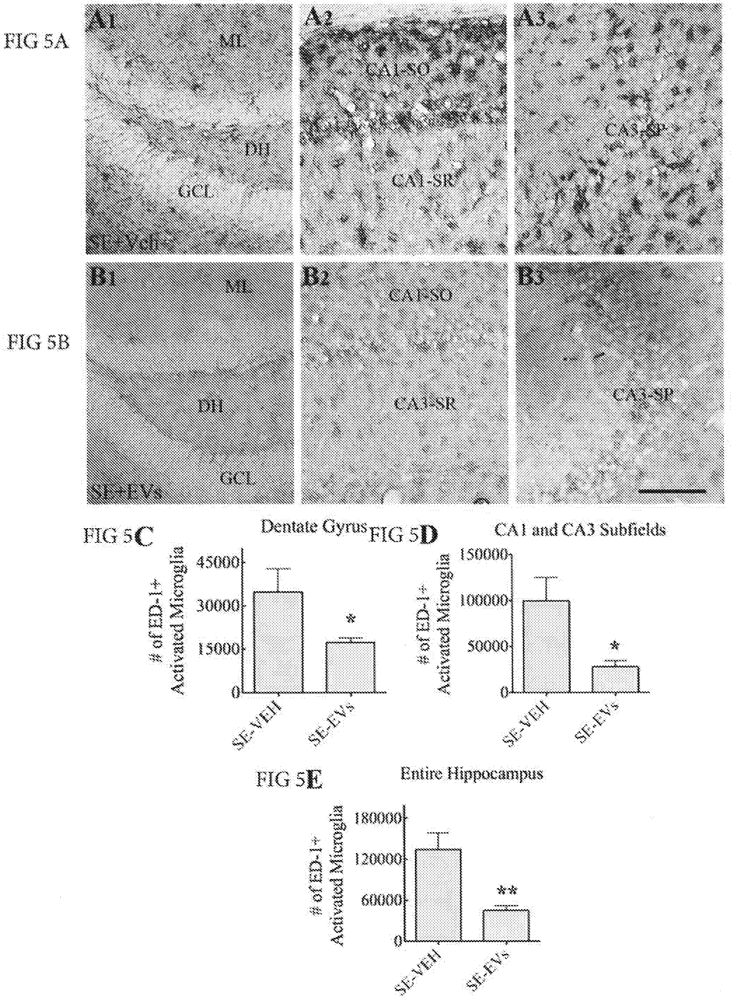

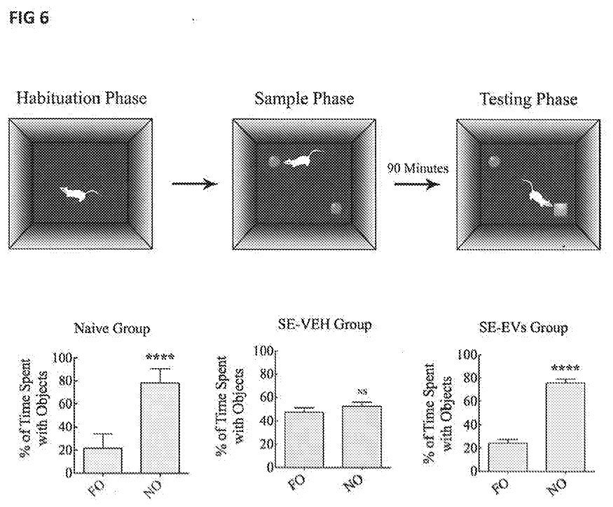

The results of this study are presented at FIG. 5.

FIG. 6 shows a significant activation of microglia (i.e. microglia expressing the protein ED-1) in the CA1 subfield of the hippocampus, in a mouse that received vehicle after SE (FIG. 5, photographs in the middle panel). In comparison, only a mild activation of microglia was observed in a mouse that received exosomes after SE (FIG. 5, photographs in the bottom panel). Top panels: examples from a naïve control mouse showing no activated microglia.

The Bar Charts in FIG. 5 (Right) illustrate the suppression of microglial activation by exosomes in the dentate gyrus, CA1 and CA3 subfield and the entire hippocampus.

### Example 7—EV A1 Preparations and Object Recognition Memory Impairment

The results of this study are presented in FIG. 6.

Recognition memory function was measured using a novel object recognition test (NORT). Certain delay was examined between the object exploration phase (which involved exploration of two identical objects for 5 minutes in an arena, i.e. “Sample Phase”) and the “Testing Phase” (which involved exploration of objects in the same arena as in the exploration phase but with replacement of one of the objects with a new object) in animals treated with EVs and animal not treated with EVs, after SE.

Mice treated with EVs after SE spent greater percentages of their object exploration time with the novel object (NO), compared to the percentage of time spent with familiar (FO) objects. In contrast, mice treated with vehicle after SE showed no ability for novel object discrimination, as they spent similar percentages of time exploring both familiar (FO) and novel (NO) objects (FIG. 6). From this data, it is demonstrated that administration of EVs after SE prevents recognition memory impairment.

### Example 8—EV A1 Preparations after SE—Averted Cognitive and Memory Impairments in the Chronic Phase

Cognitive and memory impairments typically ensue after SE.

Animals 5-6 weeks after SE with vehicle or A1-exosome treatment, via three distinct behavioral tests, were examined. These include an object location test (OLT), a novel object recognition test (NORT), and a pattern separation test (PST) (n=8-10/group).

The Object Location Test (OLT): The cognitive ability of animals was examined using an OLT. The choice to explore an object displaced to a novel location in this test reflects the ability of animal to discern minor changes in its immediate environment (FIG. 11 [A1]). Maintenance of this function depends upon the integrity of the hippocampus circuitry (32). Animals in SE-VEH group were impaired, as they did not show affinity for the object moved to a novel place (FIG. 11 [A3]). Rather, they spent nearly equal amounts of time with the object in the familiar place (FP object, FPO) and the object in the novel place (NP object, NPO, p>0.05). In contrast, animals belonging to SE-EVs group showed a greater affinity for exploring the NPO over the FPO (FIG. 11 [A4], p<0.01), which matched the normal behavior typically observed in naïve control animals (FIG. 11 [A2], p<0.0001). Since animals belonging to different groups explored objects for comparable durations (FIG. 11 [A5], p>0.05) in the testing phase, the results were not influenced by variable object exploration times between groups. Thus, SE causes hippocampus-dependent cognitive dysfunction but early intervention with A1-exosomes prevents this impairment.

The NORT Test: Recognition memory function was examined using an NORT. Recognition memory function depends upon the integrity of the perirhinal cortex and the hippocampus. Animals were examined with a 15-minute delay between the “object exploration phase” comprising the exploration of two identical objects for 5 minutes in an arena and the “testing phase” involving the exploration of objects in the same arena but with replacement of one of the objects with a new object (FIG. 11 [B1], 32). Animals belonging to SE-VEH group showed inability for novel object discrimination as they spent similar percentages of time exploring familiar and novel objects (FO and NO, FIG. 11 [B3], p>0.05). However, animals in SE-EVs group spent greater percentages of their object exploration time with the NO (FIG. 11 [B4], p<0.0001), akin to that observed in naive control animals (FIG. 11 [B2], p<0.0001). Again, animals in all groups explored objects for comparable durations (FIG. 11 [B5], p>0.05) in the testing phase. These results demonstrated that A1-exosome treatment after SE prevents recognition memory impairment.

The Pattern Separation Test: FIG. 11 presents a PST test that demonstrates that intranasal delivery of A1 exosomes after a status epilepticus (SE) episode will thwart and/or inhibit the deterioration of an animal's ability to demonstrate pattern separation, a symptom characteristic of the evolution of the SE into chronic epilepsy. The Pattern Separation Test (PST) is a relatively complex test for discriminating analogous experiences through storage of similar representations in a non-overlapping manner.

Each animal successively explored two different sets of identical objects (object types 1 and 2) placed on distinct types of floor patterns (patterns 1 and 2, P1 and P2) for 5 minutes each in the two acquisition trials separated by 15 minutes (FIG. 11 [C1]). Fifteen minutes later, in the testing phase (Trial-3), each animal explored an object from trial 2 (which is now a familiar object, FO) and an object from Trial-1 (which is now a novel object, NO) placed on the floor pattern employed in trial 2 (P2). Excellent pattern separation ability in naïve animals was revealed by a greater exploration of the object from trial 1 (i.e. NO on P2) than the object from trial 2 (i.e. FO on P2, FIG. 11 [C2], p<0.0001). In contrast, animals belonging to SE-VEH group showed no preference for the NO on P2, as they spent nearly similar amounts of time with the novel and familiar objects on P2 (FIG. 11 [C3], p>0.05), implying an impaired ability for pattern separation. However, animals in SE-EVs group spent greater percentages of their object exploration time with the novel object (FIG. 11 [C4], p<0.0001), like the behavior seen in naive control animals (FIG. 11 [C2], p<0.0001). These findings were not influenced by variable object exploration times between groups, as animals belonging to different groups explored objects for comparable durations (FIG. 11 [C5], p>0.05). Thus, A1 exosome treatment rescues animals from developing SE-induced pattern separation dysfunction.

### Example 9—EV A1 Preparations and Hippocampal Neurogenesis

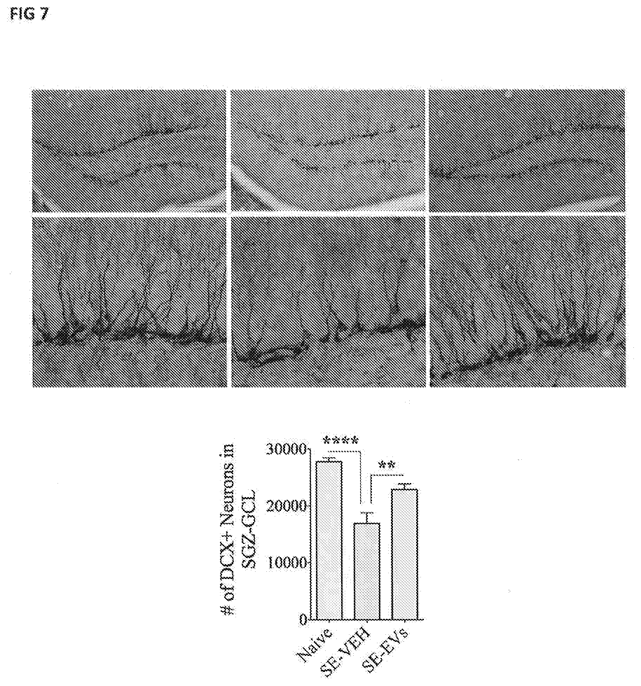

The results from this study are presented at FIG. 7.

Hippocampal neurogenesis (generation of new neurons) is one of the normal physiological events (substrates) important for maintaining normal memory function. A mouse treated with vehicle after SE demonstrates brain tissue that shows a much reduced number of newly born (double cortin expressing) neurons at ˜8 weeks after SE (FIG. 7, Top, middle two panels). However, mice treated with EVs after SE (FIG. 7, Top, far right two panels) demonstrate brain tissue evidencing the maintenance of neurogenesis to “normal” levels on control mice that have not suffered SE (naïve control mouse (FIG. 7, Top, far left two panels).

The Bar Chart (FIG. 7, bottom) compares the number of newly born neurons (double cortin (eg., “DCX”)-expressing neurons) in the three groups of mice (Naïve (Control, no SE), SE-VEH, SE-EVs). As shown, mice given EVs after SE are shown to have significantly higher levels of DCX-neurons compared to mice not given EVs (VEH (vehicle)) after SE.

This data evidences that intranasal EV administration after SE preserves near normal neurogenesis in the hippocampus.

### Example 10—Pattern Separation Test (PST) and EV Treatment

The PST test comprised three successive trials separated by 15 minute intervals following an acclimatization period of 5 minutes in an open field apparatus. The results from this test are shown in FIG. 6. The first trial comprised exploration of a pair of identical objects (type 1 objects) placed in distant areas on a floor pattern (pattern 1 or P1) for 5 minutes. The second trial involved exploration of a second pair of identical objects (type 2 objects) placed in distant areas on a different floor pattern (pattern 2 or P2) for 5 minutes. In trial 3, one of the objects from trial 2 was replaced with an object from trial 1, which became a novel object on pattern 2 (NO on P2) whereas the object retained from trial 2 became a familiar object on P2 (FO on P2). Mouse was allowed to explore objects for 5 minutes. Both trials 2 and 3 were video recorded using Noldus-Ethovision video-tracking system. Exploration of objects was defined as the length of time a mouse's nose was 1 cm away from the object area. The results such as the time spent in exploring the NO on P2 and the FO on P2 and the total object exploration time were computed from trial 3. Furthermore, NO and FO discrimination index was calculated by using the following formula: the time spent with a particular object on P2/the total object exploration time×100.

### Example 11—Preparation, Selection and Characterization of A1-Exosomes from Human Bone Marrow Derived MSCs

The generation, isolation and capturing of EVs of uniform size (80-100 nm diameter) from human bone marrow derived MSCs were performed as detailed herein. The EVs generated through this procedure were positive for classical EV markers such as CD63 and CD81 but negative for CD9 and 13 other epitopes found on the surface of MSCs. Each batch of EVs was also tested for anti-inflammatory activity in the spleen using a model of systemic inflammation induced by administration of lipopolysaccharide (LPS). Only EVs that exhibited anti-inflammatory activity in the spleen were labeled as A1-exosomes and employed in the SE model. [00127] hMSCs were obtained from the NIH-sponsored Center for the Preparation and Distribution of Adult Stem Cells (http://medicine.tamhsc.edu/irn/msc-distribution.html). The cells were from bone marrow aspirates of normal, healthy donor (donor #2015) with informed consent under Scott & White and Texas A&M Institutional Review Boards approved procedures. A frozen vial of about 1 million passage 1 hMSCs was thawed at 37° C. and plated in complete culture medium (CCM) consisting of α-minimum essential medium (α-MEM, Gibco, Grand Island, N.Y.), 17% fetal bovine serum (FBS, prescreened for rapid growth of MSCs; Atlanta Biologicals, Lawrenceville, Ga.), 100 units/ml penicillin (Gibco), 100 μg/ml streptomycin (Gibco), and 2 mM L-glutamine (Gibco) on a 152 cm2 culture dish (Corning). After 15 to 24 hours, the medium was removed, the cell layer was washed with phosphate buffered saline (PBS) and the adherent viable cells were harvested using 0.25% trypsin and 1 mM ethylenediaminetetraacetic acid (EDTA, Gibco) for 3 to 4 minutes at 37° C. The cells were re-seeded at 500 cells/cm2 in CCM and incubated for 5-7 days (with medium change on day 3) until 70 to 80% confluency (from 6,000 to 10,000 cells/cm2). The medium was removed, the cell layer washed with PBS, the cells were lifted with trypsin/EDTA and frozen at a concentration of about 1 million cells/ml in α-MEM containing 30% FBS and 5% dimethylsulfoxide (Sigma). For the experiments here, the cells were expanded under the same conditions and passage 4 cells were used.

Culture Conditions for Producing extracellular vesicles (EVs). A frozen vial of passage 4 hMSCs was thawed at 37° C. and plated directly at about 500 cells/cm2 in 150×20 mm diameter tissue culture plates (Corning 430599) in complete culture medium (CCM). The CCM medium was replaced after 2 to 3 days. After the cells reached about 70% confluency in 4 to 6 days, the medium was replaced with a medium initially optimized by supplements (58) to a commercial medium Chinese hamster ovary cells (CD-CHO Medium; cat. #10743-002; Invitrogen). The medium was recovered after 6 hours and was discarded. The medium replaced and the medium recovered between 6 and 48 hours was either stored at −80° C. or used directly to isolate EVs.

Isolation of EVs by chromatography. For isolation of EVs, the medium harvested from 40 to 45 plates (about 1.2 liters) was used directly or after thawing (20). The medium was centrifuged at 2,565×g for 15 min to remove cellular debris and the supernatant applied directly at room temperature to a column containing the anion exchange resin (100 ml bed volume; Express Q; cat. #4079302; Whatman) that had been equilibrated with 50 mM NaCl in 50 mM Tris buffer (pH 8.0). The medium was applied at a flow rate of 4 ml/min and at room temperature. The column resin was washed with 10 volumes of the equilibration buffer and then eluted with 25 volumes of 500 mM NaCl in 50 mM Tris buffer (pH 8.0). Fractions of 20 to 30 ml were collected and stored at either 4° C. or −20° C. The protein content of the EVs was assayed by the Bradford method (Bio-Rad) and the size and number by nanoparticle tracking analysis (Nanosight LM10; Malvern, Worchestershire, UK).

Assays of Anti-Inflammatory Activity of EVs. C57BL/6 male mice (Jackson Laboratories) 6 to 8 weeks old were injected through a tail vein with 150 μl of PBS, 50 μg LPS from Escherichia coli 055:B5 (Sigma, L2880) in PBS, 50 μg LPS+30 μg Dexamethasone (Sigma, D4902) in PBS, or 50 μg LPS+EVs (30 μg protein and 15 billion vesicles) in PBS. After 3 hours, the mice were killed and the spleens assayed by RT-PCR with commercial kits for IL-6, IFN-γ, and IL-10 using β-actin as an internal standard. EVs that did not produce a significant decrease (p<0.05) in all three of the pro-inflammatory factors were rejected for further use. Batches of EVs that decreased the levels of all three pro-inflammatory cytokines were chosen and referred to as A1-exosomes.

Labeling of A1-exosomes. A1-exosomes were labeled with the red fluorescent membrane dye PKH26 (Sigma, MINI26). This was done by transferring A1-exosomes from PBS to diluent C solution (Sigma) by centrifugation at 100,000×g for 70 min. PKH26, diluted to 4 mM, and the A1-exosomes (200 μg/ml) were filtered separately through small 0.2 μm syringe filters before mixing at 1:1 for 5 min, followed by the addition of 5% BSA and washing by centrifugation. The pellet of A1-exosomes was suspended in 0.5 ml PBS. To avoid dye-stained aggregates, the A1-exosomes were filtered through a 0.2 μm syringe filter immediately before use.

Animals. Male C57BL/6J mice were purchased from the Jackson Laboratory. They were 6-8 weeks old at the time of commencement of experiments. Animals were housed in an environmentally controlled room with a 12:12-hr light-dark cycle and were given food and water ad libitum. All animals were treated in accordance with a protocol approved by the Institutional Animal Care and Use Committee of Texas A&M Health Science Center College of Medicine.

Induction of Status Epilepticus (SE). Animals first received a subcutaneous (SQ) injection of scopolamine methyl nitrate (1 mg/kg, Sigma-Aldrich, S2250), as a measure to reduce the peripheral cholinergic effects of pilocarpine. Following a waiting period of 30 minutes, animals received an intraperitoneal injection of pilocarpine hydrochloride (Sigma-Aldrich, P6503) at a dose of 290-350 mg/Kg (59-61), which induced SE. Animals were closely monitored for the severity and length of the behavioral seizures. To limit the duration of SE and mortality to comparable periods, seizures were terminated in all animals with a diazepam injection (10 mg/kg, SQ.) at 2 hours after the onset of SE. The severity of convulsive responses was monitored and classified according to the modified Racine scale (62). Mice that showed consistent stage 4 (i.e. bilateral forelimb myoclonus and rearing) or stage 5 (i.e. bilateral fore- and hind-limb myoclonus and transient falling) seizures were chosen for further experimentation. Animals that did not show consistent acute seizure activity (i.e. non-responders exhibiting either no seizures or isolated milder seizures) were excluded from the study. Furthermore, animals that demonstrated extensive and severe tonic-clonic seizures (over-responders) were euthanized to avoid severe pain and distress.

Intranasal administration of A1-exosomes or vehicle. A1-exosomes were prepared using sterile PBS at a concentration of 200 μg/ml and stored at −80° C. Mice that displayed SE after a pilocarpine injection were randomly assigned to the vehicle (PBS administration, SE+Veh group) or A1-exosomes group (also referred to as SE+EVs group). Following termination of two hours of SE through a diazepam injection, each nostril was treated with 5 μl of hyaluronidase (100 U, Sigma-Aldrich, H3506) in sterile PBS to enhance the permeability of the nasal mucous membrane. Thirty minutes later, each mouse was held ventral-side up with the head facing downwards. Each nostril was then carefully administered with PBS or A1-exosomes in ˜5 μl spurts separated by 5 minutes interval, using a 10 μl micropipette. Each mouse received a total volume of 75 μl on SE day. Eighteen hours later, another 75 μl of PBS or A1-exosomes was administered in a similar manner. Overall, each mouse received a total of 150 μl of either PBS or A1 exosomes (30 μg, about 15×10⁹) within 18 hours after 2 hours of SE. However, mice in A1-exosome tracking studies received administration (75 μl) of A1-exosomes only on SE day.

Tracking of intranasally administered A-1 exosomes. For tracking intranasally administered exosomes, PKH26-labeled exosomes (15 μg, about 7.5 billion per mouse) were administered only on SE day, using methods described above (n=4). Each mouse was transcardially perfused with saline and paraformaldehyde (PH=7.4) at 6 hours following A1-exosome administration. The brains were removed, post-fixed in 4% paraformaldehyde overnight, and cryoprotected with different grades of sucrose solution. Thirty-micrometer thick coronal sections were cut through the entire brain using a cryostat and the sections were collected serially in 24-well plates containing phosphate buffer (PB). Representative sets of sections (every 10^{th}) through different levels of the cortex and the hippocampus were chosen for tracking the intranasally administered A1-exosomes through dual immunofluorescence and confocal microscopy. Briefly, different sets of sections were labelled with primary antibodies for NeuN (a pan neuronal marker, Millipore, ABN78), GFAP (a marker of astrocytes, Millipore, MAB360), or IBA-1 (a marker of microglia, Abcam, ab5076). The secondary antibodies comprised Cy2 conjugated donkey anti-goat IgG (Jackson Immuno Research, 715-225-150), Cy2 conjugated donkey anti-rabbit IgG (Jackson Immuno Research, 711-545-152) or A488 anti-mouse IgG (Thermo Fisher Scientific, A-21202). Sections were mounted using an antifade reagent (Sigma, S7114). One μm-thick optical Z-sections were sampled from different regions of the cortex and various subfields of the hippocampus using a confocal microscope (FV10i, Olympus or Ti-Eclipse, Nikon) and the images were analyzed using Olympus FV-10i image browser.

Preparation of hippocampal extract and evaluation of cytokines. Twenty four hours after the first administration of PBS or A1-exosomes (i.e. 75 μl administration performed ˜2 hours after SE induction), mice were anesthetized with isoflurane and the brains were rapidly dissected from the skull and stored at −80° C. (n=6/group). For comparison, brains were similarly collected from naïve control animals (n=6). For the evaluation of cytokine levels in the hippocampus, each brain was thawed, the hippocampus was rapidly dissected under a stereomicroscope and sonicated on ice in lysis buffer containing protease inhibitor cocktail (Sigma, P2714), and centrifuged at 10,000 RPM for 5 minutes at 4° C. The supernatant was collected, the total protein concentration was measured and the lysate was diluted for the required concentration. Each 96-well cytokine array plate (Signosis, EA-4005) used in this study displayed 4 segments (24 wells/segment) adequate for measuring 24 different cytokines from four samples. The wells were pre-coated with specific cytokine capture antibodies. The assay was performed as per the manufacturer's guidelines with each well receiving 10 μg of lysate (in 100 μl volume). In this assay, the concentration of each of 24 cytokines in hippocampal lysates is directly proportional to the intensity of color. For TNF-α (Signosis, EA-2203) and IL1-β (Signosis, EA-2508) enzyme-linked quantitative immunoassays, 100 μl of serially diluted standard and 100 μl hippocampal lysate were used. The assay was performed as per the manufacturer's guidelines. The levels of TNF a and IL1-β were quantified using the standard graph and expressed as pg/mg of protein.

Tissue processing and immunohistochemistry. Four days after SE, subgroups of animals belonging to both SE+Veh and SE+EVs group (n=6/group) were perfused with 4% paraformaldehyde, as described in the earlier section. A subgroup of age-matched naïve control animals (n=5) were also perfused similarly. Several sets of serial sections (every 15^{th} or 20^{th}) through the entire hippocampus were selected and processed for immunohistochemistry. The procedures employed for immunostaining are described in our previous reports (5). Briefly, the sections were etched with PBS solution containing 20% methanol and 3% hydrogen peroxide for 20 minutes, rinsed thrice in PBS, treated for 30 minutes in PBS containing 0.1% Triton-X 100 and an appropriate serum (10%) selected on the basis of the species in which the chosen secondary antibody was raised. The primary antibodies comprised anti-CD68 (ED-1, an activated microglia marker; Bio-Rad Laboratories, MACA341R), anti-NeuN (a pan neuronal marker; Millipore, ABN78), anti-parvalbumin (PV, a calcium binding protein found in a subclass of GABA-ergic interneurons; Sigma-Aldrich, P3088), anti-somatostatin (SS, a neuropeptide found in a subclass of GABA-ergic interneurons; Peninsula Laboratories, T-4546) or anti-neuropeptide Y (NPY, another neuropeptide found in a subclass of GABA-ergic interneurons; Peninsula Laboratories, T-4070). After an overnight incubation with the respective primary antibody solution, sections were washed thrice in PBS and incubated in an appropriate secondary antibody solution for an hour. Biotinylated anti-rabbit (H+L) (Vector Lab, BA-1000) and biotinylated anti-mouse (H+L) (Vector Lab, BA-2000) were used for the study. The sections were washed thrice in PBS and treated with avidin-biotin complex reagent (Vector Lab, PK-6100) for an hour. Peroxidase reaction was developed using diaminobenzidine (Vector Lab, SK-4100) or vector SG (Vector Lab, SK-4700) as chromogens, and the sections were mounted on gelatin coated slides, dehydrated, cleared and cover slipped with permount.

Measurement of the number of various immunostained cells. The optical fractionator method in the StereoInvestigator system (Microbrightfield Inc., Williston, Vt.) interfaced with a Nikon E600 microscope through a color digital video camera (Optronics Inc., Muskogee, Okla.) was employed for all cell counts performed at 4 days post-SE. This comprised quantification of numbers of: (i) activated microglia positive for ED-1 in the dentate gyrus (DG) and CA1 and CA3 subfields; (ii) neurons positive for NeuN in the dentate hilus (DH) and the CA1 pyramidal cell layer; (iii) interneurons positive for PV, SS and NPY in the DH+granule cell layer (GCL) and CA1 and CA3 subfields. A detailed methodology employed in these counts is described in our previous report (5,63).

Behavioral tests. Animals were examined with three object based tests at 5-6 weeks after SE in SE+Veh and SE+EVs groups (n=8-10/group). Age-matched naïve control animals (n=10) were also included for comparison. The tests comprised an object location test (OLT), a novel objection recognition test (NORT) and a pattern separation test (PST). All tests were performed using an open field apparatus (measuring 45×45 cm).

Object location test (OLT). Each mouse was observed in an open field with three successive trials separated by 15 minute intervals. A detailed description of this test is available in our previous report (32). In brief, the mouse was placed in an open field for 5 minutes in the first trial for acclimatization to the testing apparatus (habituation phase) whereas in trial 2, the mouse was allowed to explore two identical objects placed in distant areas of the open field (sample phase). In trial 3 (testing phase), one of the objects was moved to a new area (novel place object, NPO) while the other object remained in the previous place (familiar place object, FPO). Both trials 2 and 3 were video recorded using Noldus-Ethovision video-tracking system to measure the amount of time spent with each of the two objects. Exploration of the object was defined as the length of time a mouse's nose was 1 cm away from the marked object area. The results such as the percentage of object exploration time spent in exploring the NPO and FPO as well as the total object exploration time in trial 3 were computed. The percentage of time spent with the NPO and FPO was calculated by using the following formula: the time spent with the particular object/the total object exploration time×100.

Novel object recognition test (NORT). Each mouse was examined in an open field with three consecutive trials separated by 15 minute intervals. A detailed description of this test is available in our previous report (32). The first two trials comprised an acclimatization period of 5 minutes to an open field (trial 1 or habituation phase) and exploration of two similar objects placed in distant areas within the open field for 5 minutes (sampling phase). In trial 3 (testing phase), the mouse was allowed to explore a different pair of objects comprising one of the objects used in trial 2 and a novel object for 5 minutes. Both trials 2 and 3 were video recorded using Noldus-Ethovision video-tracking system. The amounts of time spent with the familiar object area (FOA) and the novel object area (NOA) and the total object exploration time in trial 3 were computed. Exploration of the FOA or NOA was defined as the length of time a mouse's nose was 1 cm away from the respective area. The percentages of time spent with the NOA or FOA were calculated by using the following formula the time spent with the particular object/the total object exploration time×100.

Pattern separation test (PST). This test comprised three successive trials separated by 15 minute intervals following an acclimatization period of 5 minutes in an open field apparatus. The first trial comprised exploration of a pair of identical objects (type 1 objects) placed in distant areas on a floor pattern (pattern 1 or P1) for 5 minutes. The second trial involved exploration of a second pair of identical objects (type 2 objects) placed in distant areas on a different floor pattern (pattern 2 or P2) for 5 minutes. In trial 3, one of the objects from trial 2 was replaced with an object from trial 1, which became a novel object on pattern 2 (NO on P2) whereas the object retained from trial 2 became a familiar object on P2 (FO on P2). Mouse was allowed to explore objects for 5 minutes. Both trials 2 and 3 were video recorded using Noldus-Ethovision video-tracking system. Exploration of objects was defined as the length of time a mouse's nose was 1 cm away from the object area. The results such as the time spent in exploring the NO on P2 and the FO on P2 and the total object exploration time were computed from trial 3. Furthermore, NO and FO discrimination index was calculated by using the following formula: the time spent with a particular object on P2/the total object exploration time×100.

Analyses of hippocampal neurogenesis and inflammation in the chronic phase after SE. Animals were perfused 6 weeks post-SE using 4% paraformaldehyde. Tissue processing, section cutting and storage of sections were done as described earlier. Serial sections (every 10^{th}) through the entire hippocampus were selected from animals belonging to naïve control, SE+Veh and SE+EVs groups (n=6/group) and processed for immunohistochemistry. The detailed procedures employed for immunostaining are available in our previous report (5). The primary antibodies comprised anti-doublecortin (anti-DCX, a marker of newly born neurons [63]; Santa Cruz Biotechnology, sc-8066; Abcam, ab5076), anti-reelin (marker of a subclass of interneurons that secrete reelin in the hippocampus; Millipore, MAB5364), Prox-1 (a marker of dentate granule cells; Millipore, AB5475) and anti-IBA-1 (a pan microglial marker; Abcam, ab5076). The secondary antibodies comprised biotinylated anti-goat (H+L) (Vector Lab, BA-9500), biotinylated anti-mouse (H+L) (Vector Lab, BA-2001), or biotinylated anti-rabbit (H+L) (Vector Lab, BA-1000). Following incubation in secondary antibody solutions, sections were incubated with avidin-biotin complex reagent (Vector Lab, PK-6100). The peroxidase reaction was developed using diaminobenzidine (Vector Lab, SK-4100) or vector SG (Vector Lab, SK-4700) as chromogens, and the sections were mounted on gelatin coated slides, dehydrated, cleared and cover slipped with permount.

Quantification of numbers of DCX+, prox-1+ and reelin+ neurons. An optical fractionator method available in the StereoInvestigator system (Microbrightfield Inc.) was employed for counting: (i) newly born neurons positive for doublecortin (DCX) in the subgranular zone-granule cell layer (SGZ-GCL) of the hippocampus; (ii) prox-1+ granule cells in the DH; and (iii) reelin+ interneurons in the DH. Quantification was done from 5-6 animals per group. A detailed methodology employed in these counts is described in a previous report (5,63).

Measurement of the area fraction of IBA-1+ immunoreactive elements. The area occupied by IBA-1+ immunoreactive elements (soma and processes of microglia) in the DG and CA1 and CA3 subfields were quantified using Image J software, as described in our previous report (5). In brief, images from different regions of the hippocampus were digitized using a 20× objective lens in a Nikon E600 microscope equipped with a digital video camera connected to a computer. Each image saved in gray scale as a bitmap file was opened in Image J software, and a binary image was created through selecting a threshold value that retained all IBA-1+ structures but no background. The area occupied by the IBA-1+ structures (i.e. the area fraction) in the binary image was then measured by selecting the Analyze command in the program. Area fraction of IBA-1+ immunoreactive elements was calculated separately for every hippocampal region in each animal by using data from all chosen serial sections before the mean and SEM were determined for the total number of animals included per group (n=4/group).

Statistical analysis. Statistical analyses were performed using Prism software. One-way analyses of variance (one-way ANOVA) with Newman-Keuls multiple comparison post hoc tests was employed when 3 groups were compared. Comparison within groups in the behavioral tests or comparison between the two groups (e.g. prox-1 counts) employed unpaired, two-tailed Student's-t test. Numerical data were presented as mean±SEM and a p value less than 0.05 was considered as statistically significant.

### Example 12—Intranasally Dispensed A1-Exosomes Incorporated into Cortical and Hippocampal Neurons

IN administration of A1-exosomes after SE was examined to determine if this would result in targeting of these exosomes into the hippocampus, the region exhibiting intense hyperactivity of neurons, increased oxidative stress and inflammation with infiltration of peripheral monocytes during and/or after SE (5, 12). Intranasally administered PKH26-labeled A1-exosomes (15 μg, ˜7.5×10⁹) was provided immediately after the termination of 2 hours of SE by an injection of diazepam. Six hours later, animals were perfused (n=4) and serial sections through the entire brain were processed for immunofluorescence using markers of neurons (neuron-specific nuclear antigen, NeuN), astrocytes (glial fibrillary acidic protein, GFAP), and microglia (IBA-1) and Z-sectioning in a confocal microscope.

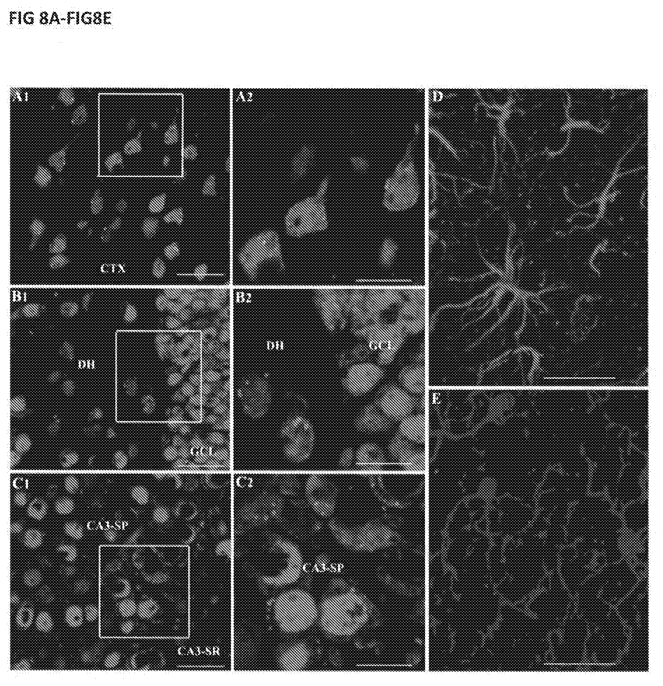

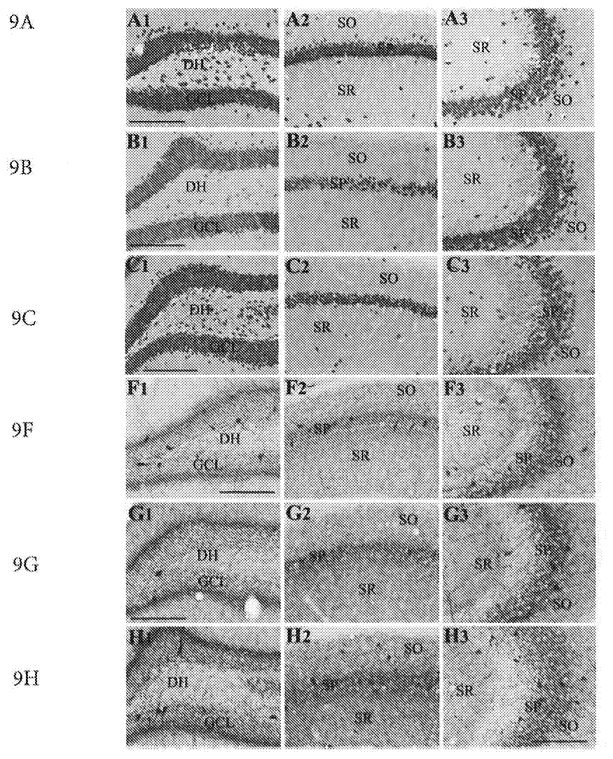

Red colored PKH26+ particles (i.e. A1-exosomes) were found throughout the olfactory bulb, fronto-parietal cortex, basal forebrain, striatum and the dorsal hippocampus. At dorsal hippocampal levels, most exosomes were in smaller clusters and were seen either within the cytoplasm of neurons or attached to the cell membrane of neurons (FIG. 8 [A1-C2]). In the hippocampus, exosomes were clearly seen within dentate hilar neurons (FIG. 8 [B1, B2]) and the CA3 pyramidal neurons (FIG. 8 [C1, C2]). Occasionally, exosomes were also found in the cytoplasm of dentate granule cells (FIG. 8 [B1, B2]) and the CA1 pyramidal neurons. Tissues were also examined for exosome presence within GFAP+ astrocytes (FIG. 8 [D]) and IBA-1+ microglial cells (FIG. 8 [E]) in the hippocampus. None were seen in the cell body of astrocytes but were found inside the cell body of some microglia. Exosomes were however frequently seen in close proximity to astrocyte and microglial processes. In rostral regions of the cerebral cortex, accumulation of exosomes could be seen in virtually all neurons and a vast majority of microglia (see FIGS. 13 and 14). Exosomes were also seen in close proximity to processes of astrocytes and microglia. Interestingly, while neurons displayed either isolated or smaller clusters of exosomes, a greater fraction of microglia displayed larger clusters of exosomes within their cytoplasm (see FIG. 14). Thus, within 6 hours of IN administration, A1-exosomes incorporated robustly into neurons and microglia in rostral regions of the cerebral cortex, and predominantly into neurons in the cortex and the hippocampus at dorsal hippocampal levels.

### Example 13—IN Delivery of A1-Exosomes after SE Prevented the Rise of Multiple Pro-Inflammatory Cytokines and Increased the Concentration of Some Anti-Inflammatory Cytokines and Growth Factors in the Hippocampus

Twenty four (24) cytokines in hippocampal lysates obtained from animals belonging to different groups (n=6/group) at 24 hours post-SE, using 96-well array plates that were pre-coated with specific cytokine capture antibodies. Sixteen pro-inflammatory cytokines exhibited upregulation in animals receiving vehicle after SE (SE-VEH group), in comparison to naïve control animals.

The concentration of 7 pro-inflammatory cytokines was significantly reduced in animals receiving A1-exosomes after SE (SE-EVs group, FIG. 2 [A-G]) in comparison to animals in SE-VEH group. These include tumor necrosis factor-alpha (TNF-α), interleukin-1 β (IL1-β), monocyte chemoattractant protein-1 (MCP-1), stem cell factor (SCF), macrophage inflammatory protein-1 alpha (MIP-1α), granulocyte-macrophage colony-stimulating factor (GM-CSF), and interleukin-12 (IL-12). Animals in SE-EVs group also displayed enhanced concentrations of anti-inflammatory cytokine interleukin-10 (IL-10, FIG. 2 [H]), granulocyte colony stimulating factor (G-CSF, FIG. 2 [I]), platelet derived growth factor-β (PDGF-B, FIG. 2 [J]), interleukin-6 (IL-6, FIG. 2 [K]), and interleukin-2 (IL-2, FIG. 2 [L]). Since TNF-α and IL1-β are among the major pro-inflammatory cytokines that are implicated in brain diseases exhibiting inflammation and/or cognitive and memory dysfunction and have pro-convulsive properties (31), the concentration of these agents was further confirmed through independent quantitative ELISAs.

The results clearly showed their upregulation at 24 hours post-SE in animals belonging to SE-VEH group and normalized levels in SE-EVs group (FIG. 2 [M,N]). Thus, IN administration of A1-exosomes commencing 2 hours post-SE was adequate for greatly easing the inflammatory storm triggered by SE.

### Example 14—IN Delivery of A1-Exosomes after SE Greatly Reduced the Activation of Microglia in the Hippocampus

The extent of inflammation in the hippocampus at 4 days post-SE in animals receiving vehicle or A1-exosomes was measured through immunohistochemical staining of serial sections for ED-1 (CD68, a marker of activated microglia or macrophages in the brain) and stereological quantification of ED-1+ cells in the dentate gyrus (DG) and CA1 and CA3 subfields of the hippocampus (FIG. 5E) (n=5-6/group). Animals in SE-VEH group displayed increased density of ED-1+ microglia with several morphological changes particularly in the CA1 and CA3 panel subfields (FIG. 5D).

A fraction of microglia exhibited hypertrophy of soma with multiple short processes while some others displayed round or oval shaped soma with no or minimal processes, both of which are characteristics of activated microglia. In contrast, animals in SE-EVs group not only displayed reduced density of ED-1+ microglia but also a greatly diminished intensity of ED-1 staining (FIG. 5A [B1-B3]). Stereological quantification confirmed reduced numbers of ED-1+ microglia in the DG CA1 and CA3 subfields (FIG. 5A), and in the entire hippocampus (FIG. 5 [E]). The reductions were 50% for the DG, 72% for the CA1 and CA3 subfields and 66% for the entire hippocampus (p<0.05-0.01, FIG. 5 [C-E]).

### Example 15—IN Delivery of A1-Exosomes after SE Reduced the Overall Loss of Neurons in the Dentate Hilus and the CA1 Cell Layer of the Hippocampus

SE typically causes degeneration of neurons in certain regions/layers of the hippocampus. To ascertain the extent of SE-induced neurodegeneration in the hippocampus of animals receiving vehicle or A1-exosomes after SE, NeuN immunostaining of serial sections through the entire hippocampus was performed at 4 days post-SE (FIG. 9A [Panels A1-C3]). In comparison to naïve control animals, both SE-VEH and SE-EVs groups showed reduced densities of neurons in the dentate hilus (DH) and the CA1 pyramidal cell layer but no discernable changes in the granule cell layer (GCL) and the CA3 pyramidal cell layer (FIG. 9A [Panels A1-C3], n=5-6/group). Stereological quantification revealed that the overall neuron loss in the DH and CA1 cell layer ranged from 40-47% in the SE-VEH group (p<0.001) and 25-26% in the SE-EVs group (p<0.01-0.001). Because of neuroprotection mediated by A1-exosomes, animals in SE-EVs group displayed 30-41% greater number of neurons than animals in the SE-VEH group (p<0.01-0.001, FIG. 9D-FIG. 9E]). Thus, IN administration of A1-exosomes after SE reduced the loss of neurons in regions of the hippocampus that are highly susceptible to SE-induced neurodegeneration.

### Example 16—IN Delivery of A1-Exosomes after SE Restrained the Loss of Several Subclasses of Inhibitory Interneurons in the Hippocampus

Several subclasses of inhibitory GABA-ergic interneurons in the hippocampus are highly susceptible to SE. To measure the extent of SE-induced loss of inhibitory interneurons in animals receiving vehicle or A1-exosomes after SE, immunostaining of serial sections was performed through the entire hippocampus for the calcium binding proteins parvalbumin (PV), and neuropeptides somatostatin (SS) and neuropeptide Y (NPY) at 4 days post-SE (n=5-6/group). The interneurons positive for PV displayed reduced density in the dentate hilus-granule cell layer (DH-GCL) region and the CA1 subfield after SE (FIG. 9A [Panels F1-H3]). Stereological measurement demonstrated that the overall PV+ interneuron loss in the DH-GCL and the CA1 subfield varied from 43-56% in the SE-VEH group (p<0.001) and 24-25% in the SE-EVs group (p<0.01-0.001). Due to the protection mediated by A1-exosomes, the SE-EVs group displayed 34-69% greater numbers of PV+ interneurons than the SE-VEH group (p<0.05, FIG. 9I, FIG. 9J). Interneurons expressing SS exhibited reduced densities in the DH+GCL, CA1 and CA3 regions after SE (FIG. 10A [Panels A1-C3]). Stereological cell counting showed that the overall SS+ interneuron loss in these regions ranged from 39-44% in the SE-VEH group (p<0.01-0.001). In contrast, the SE-EVs group displayed no significant loss in the DH+GCL region and the CA1 subfield (p>0.05) but 27% loss in the CA3 subfield (p<0.05). In comparison to the SE-VEH group, the SE-EVs group displayed 47-52% greater numbers of SS+ interneurons in the DH+GCL and CA1 regions (p<0.01, FIG. 10E) and 20% higher number in the CA3 subfield (p>0.05, FIG. 10F). The interneurons positive for NPY displayed reduced density only in the DH-GCL region after SE (FIG. 10A [Panels G1-I3]). Stereological quantification revealed that the NPY+ interneuron loss in the DH+GCL region is 46% in the SE-VEH group and 35% in the SE-EVs group (p<0.01, FIG. 10J). In comparison to the SE-VEH group, the SE-EVs group displayed 22% higher numbers of NPY+ interneurons (p>0.05, FIG. 10J). Thus, IN administration of A1-exosomes after SE diminished the loss of several subclasses of GABA-ergic interneurons in the hippocampus.

### Example 17—IN Delivery of A1-Exosomes after SE Promoted Normal Hippocampal Neurogenesis in the Chronic Phase

Hippocampal neurogenesis exhibits a biphasic response to SE, with increased and abnormal neurogenesis in the early phase and persistently declined neurogenesis in the chronic phase (6, 7). The effects of IN administration of A1-exosomes was examined after SE on long-term neurogenesis in the hippocampus (i.e. 6 weeks after SE, n=6/group). In comparison to naïve controls (FIG. 12A1, 12A2]), animals in SE-VEH group demonstrated decreased neurogenesis (FIG. 12B1, 12B2], p<0.0001) whereas animals in SE-EVs group (FIG. 12C1, 12C2]), displayed a pattern and extent of neurogenesis that is equivalent to age-matched naïve control animals (p>0.05) and greater extent of neurogenesis than animals in SE-VEH group (p<0.01, FIG. 12D). Furthermore, SE-VEH animals showed significant loss of dentate hilar neurons positive for reelin, a protein important for directing the migration of newly born neurons in the subgranular zone (SGZ) to the GCL (FIG. 12E-12H], p<0.01). Interestingly, reelin+ positive neuron numbers in SE-EVs group were comparable to naïve control animals (FIG. 12 [E-12H], p>0.05) and greater than SE-VEH group (p<0.05). To determine the extent of abnormal migration of newly born granule cells into the dentate hilus, the numbers of neurons positive for prox-1 (a marker of dentate granule cells) in the dentate hilus (FIG. 12I-12L]) were quantified. This revealed reduced abnormal migration of newly born granule cells into the dentate hilus in animals belonging to SE-EVs group, in comparison to SE-VEH group (p<0.05, FIG. 12L). Thus, A1-exosome treatment after SE facilitated maintenance of normal pattern and extent of neurogenesis with preservation of reelin+ neurons and minimal aberrant migration of newly born granule cells.

### Example 18—IN Delivery of A1 Exosomes after SE LED to Reduced Hippocampal Inflammation in the Chronic Phase

To examine whether A1-exosome mediated suppression of hippocampal inflammation observed in the early phase after SE persists in the chronic phase, microglia in the hippocampus was examined through IBA-1 immunostaining 6 weeks after SE (FIG. 12M1-12Q]). Animals in SE-VEH group demonstrated enhanced density of microglia with hypertrophied soma and thick, short processes (FIG. 12N1-12N3]). Such microglia were prominently seen in the DG and the CA1 subfield. In contrast, animals in SE-EVs group showed highly ramified microglia (FIG. 12O1-12O3), akin to that seen in age-matched naïve control animals (FIG. 12M1-12M3]). Measurement of the area occupied by IBA-1 reactive elements revealed increased microglial activity in DG and CA1 regions of animals belonging to SE-VEH group, in comparison to both naïve control group and SE-EVs group (FIG. 12P-12Q], p<0.001, n=4/group). Thus, A1-exosome treatment early after SE restrained hippocampal inflammation for prolonged periods.

### Example 19—Pharmaceutical Preparation of A1 Extracellular Vesicles and/or Exosomes

The present example describes a pharmaceutical preparation provided as a pre-banked extracellular vesicle (EVs) preparation that may be stored until needed for use. The A1 EVs will be isolated from cell culture medium in which mesenchymal stem cells, such as human or non-human mesenchymal stem cells (MSCs), have been cultured for an appropriate period of time.

A sub-population of A1-exosomes screened from a population of mixed exosomes harvested from cell culture medium in which mesenchymal stem cells have grown may be provided. The selected sub-population of A1 exosomes will be screened and selected for those having a defined enhanced baseline anti-inflammatory and neuroprotective activity, compared to other exosomes present in the cell media. As a selection criteria, exosomes that are selected as A1 exosomes will be identified that increase the expression of IL-10, G-CSF, PDGF-β, IL-6, IL-2, or any combination of two or more of these. The A1 exosomes may further be selected based on size.

Therapeutic benefits of A1 EV administration include paracrine effects mediated by soluble factors. The EVs are able to cross the blood-brain barrier and thereby deliver various therapeutic factors to the brain. The A1 EVs of the present preparations also may contain a multitude of mRNAs, miRNAs and proteins that can be harvested, characterized and banked isolated from the A1 EVs. The A1 EVs mat be secreted by MSCs obtained from several sources such as bone marrow, lipoaspirate of liposuction procedures, umbilical cord and human induced pluripotent stem cells. The use of the herein described A1 EVs avoids several potential safety hazards attendant other alternative cell therapies, such as the risk of tumors. Hence, these as well as other advantages are provided over use of cell containing approaches for use in clinical therapies. The present A1 exosomes compositions are readily defined and standardized since they are stable and not responsive to external stimuli. In addition, the A1 exosomes can be made readily available for use in patients as they are far more stable to freezing and thawing.

The efficacy of IN administration of the A1 exosomes and EVs derived from human bone marrow derived MSCs was demonstrated here using a pilocarpine model of SE in mice. The EVs used as part of the present preparations are well characterized and are referred to as A1-exosomes because of their demonstrated robust anti-inflammatory properties, as confirmed by particular screening tools to select for such populations of EVs having a higher anti-inflammatory activity compared to other EV preparations produced by MSCs.

Alternatively, an A1-exosome population of EVs may be selected based on the ability of the EVs to satisfy two or more of the following criteria:

1. Ability to enter the hippocampus,

2. Ability to suppress inflammation, as determined by anti-inflammatory cytokine activity for suppressing IL1-β and TNF-α,

3. Ability to protect glutamatergic and gamma-amino butyric acid-ergic (GABA-ergic) neurons in the early phase after SE was measured,

4. Ability to enhance anti-inflammatory cytokines such as IL-10.

It is specifically intended that the present invention not be limited to the embodiments and illustrations contained herein, but include modified forms of those embodiments including portions of the embodiments and combinations of elements of different embodiments as come within the scope of the following claims.

### BIBLIOGRAPHY

The following references are specifically incorporated herein by reference.
1. Hattiangady, B., et al., Neurobiol. Dis. 17,473-490 (2004).
2. Hattiangady, B. & Shetty, A. K. Hippocampus 20, 97-112 (2010).
3. Ben-Ari, Y. in Jasper's Basic Mechanisms of the Epilepsies (eds Noebels, J. L., Avoli, M., Rogawski, M. A., Olsen, R. W. & Delgado-Escueta, A. V.) (Michael A Rogawski, et al., Bethesda (Md.), 2012).
4. Kleen, J. K., et. al., in Jasper's Basic Mechanisms of the Epilepsies (eds Noebels, J. L., et al.) (Michael A Rogawski, et al., Bethesda (Md.), 2012).
5. Loscher, W. in Jasper's Basic Mechanisms of the Epilepsies (eds Noebels, J. L., et. al.) (Michael A Rogawski, et al., Bethesda (Md.), 2012).
6. Sankar, R. & Mazarati, A. in Jasper's Basic Mechanisms of the Epilepsies (eds Noebels, J. L., Avoli, M., Rogawski, M. A., Olsen, R. W. & Delgado-Escueta, A. V.) (Michael A Rogawski, et al., Bethesda (Md.), 2012).
7. Fellin, T. & Haydon, P. G. Trends Mol. Med. 11, 530-533 (2005).
8. Vezzani, A., French, J., Bartfai, T. & Baram, T. Z., Nat. Rev. Neurol. 7, 31-40 (2011).
9. Parent, J. M. et al. J. Neurosci. 17, 3727-3738 (1997).
10. Shetty, A. K. & Hattiangady, B. Stem Cells 25, 2396-2407 (2007).
11. Scharfman, H. E. & Pierce, J. P. Epilepsia 53 Suppl 1, 109-115 (2012).
12. Temkin, N. R. Epilepsia 42, 515-524 (2001).
13. Temkin N. R., Epilepsy Res 60, 80-83 (2004).
14. Dichter, M. A., Epilepsia 50 Suppl 2, 41-45 (2009).
15. Seinfeld S, et al. (2016) Status epilepticus. Cold Spring Harb Perspect Med 6(3):a022830.
16. Khoujah D, Abraham M K (2016), Emerg Med Clin North Am 34(4):759-776.
17. Vezzani A, Dingledine R, Rossetti A O (2015), Expert Rev Neurother 15(9):1081-1092.
18. Loscher W (2015), Epilepsy Behav 49:20-25.
19. Mishra V. et al. (2015), Sci Rep 5:17807.
20. Shetty A K, Hattiangady B (2007) Stem Cells 25(10):2396-2407.
21. Hattiangady B, Shetty A K (2010), Hippocampus 20(1):97-112.
22. Ben-Ari Y (2012), Epilepsia 53(2):394-396.
23. Loscher W (2012), Jasper's Basic Mechanisms of the Epilepsies, eds Noebels J L, Avoli M, Rogawski M A, Olsen R W, Delgado-Escueta A V (Michael A Rogawski, et al., Bethesda Md.), pp. 1055-1065.
24. Sankar R, Mazarati A (2012), Jasper's Basic Mechanisms of the Epilepsies, eds Noebels J L, Avoli M, Rogawski M A, Olsen R W, Delgado-Escueta A V (Michael A Rogawski, et al., Bethesda Md.), pp. 945-956.
25. Shetty A K (2014), Epilepsy Behav 38:117-124.
26. Varvel N H, et al. (2016), Proc Natl Acad Sci USA 113(38):E5665-5674.
27. Sahin M, Menache C C, Holmes G L, Riviello J J (2001), Epilepsia 42(11):1461-1467.
28. Temkin N R (2001, Epilepsia 42(4):515-524.
29. Dichter M A (2009), Arch Neurol 66(4):443-447.
30. Trinka E, Kalviainen R (2016). Seizure.
31. Sloviter R S, Bumanglag A V (2013), Neuropharmacology 69:3-15.
32. Patterson V, Pant P, Gautam N, Bhandari A (2014) Seizure 23(7):567-569.
33. Agadi S, Shetty A K (2015). Stem Cells 33(7):2093-2103.
34. Kim D K, et al. (2016a). Proc Natl Acad Sci USA 113(1):170-175.
35. Salem H K, Thiemermann C (2010). Stem Cells 28(3):585-596.
36. Phinney D G, et al. (2015), Nat Commun 6:8472.
37. Heldring N, et al., (2015). Hum Gene Ther 26(8):506-517.
38. Anderson J D, et al. (2016), Stem Cells 34(3):601-613.
39. Cossetti C. et al. (2014). Mol Cell 56(2):193-204.
40. Batrakova E V, Kim M S (2015). J Control Release 219:396-405.
41. S ELA, et al., (2013). Nat Rev Drug Discov 12(5):347-357.
42. Kimbrel E A, et al. (2014). Stem Cells Dev 23(14):1611-1624.
43. Watson N. et al. (2015). Cytotherapy 17(1):18-24.
44. Lim M H, Ong W K, Sugii S (2014). Expert Rev Mol Med 16:e8.
45. Li G, et al. (2011) Cytokines and epilepsy. Seizure 20(3):249-256.
46. Hattiangady B, et al. (2014), Front Behav Neurosci 8:78.
47. Yassa M A, Stark C E (2011). Trends Neurosci 34(10):515-525.
48. Leutgeb J K, Leutgeb S, Moser M B, Moser E I (2007). Science 315(5814):961-966.
49. Wasterlain C G, et al., (2013) Epilepsia 54 Suppl 6:78-80.
50. Janigro D, Iffland P H, 2nd, Marchi N, Granata T (2013), Epilepsia 54 Suppl 6:30-32.
51. Amor M M, et al., (2014). Case Rep Crit Care 2014:918247.
52. Vezzani A (2013). Ann Neurol 74(1):1-3.
53. Prinz M, Priller J (2014). Nat Rev Neurosci 15(5):300-312.
54. Rao M S, Hattiangady B, Reddy D S, Shetty A K (2006). J Neurosci Res 83(6):1088-1105.
55. Hattiangady B, Kuruba R, Shetty A K (2011). Aging Dis 2(1):1-17.
56. Vezzani A, Sperk G, Colmers W F (1999). Trends Neurosci 22(1):25-30.
57. Schwaller B. et al. (2004). Mol Cell Neurosci 25(4):650-663.
58. Houser C R (2014) Adv Exp Med Biol 813:151-160.
59. Murray A J, et al. (2011). Nat Neurosci 14(3):297-299.
60. Parent J M. et al. (1997). J Neurosci 17(10):3727-3738.
61. Hattiangady B, Rao M S, Shetty A K (2004). Neurobiol Dis 17(3):473-490.
62. Cho K O. et al. (2015). Nat Commun 6:6606.
63. Kuruba R, Hattiangady B, Shetty A K (2009). Epilepsy Behav 14 Suppl 1:65-73.
64. Scharfman H E, Goodman J H, Sollas A L (2000). J Neurosci 20(16):6144-6158.
65. Scharfman H E, et al., (2002). J Comp Neurol 454(4):424-439.
66. Scharfman H E, et al., (2003). J Neurophysiol 90(4):2536-2547.
67. McCloskey D P, et al., (2006). Eur J Neurosci 24(8):2203-2210.
68. Hester M S, Danzer S C (2014) Epilepsy Behav 38:105-116.
69. Gong C, Wang T W, Huang H S, Parent J M (2007). J Neurosci 27(8):1803-1811.
70. Ryan S M, Nolan Y M (2016) Neurosci Biobehav Rev 61:121-131.
71. Mendelsohn A R, Larrick J (2016), Rejuvenation Res. 19(6):521-524.
72. Kim D K et al. (2016a), PLoS One 11(1):e0147553.
73. Cunningham M. et al. (2014), Cell stem cell 15(5):559-573.
74. Chen X, et al. (2016), Epilepsy Res 119:24-29.
75. Shibley H & Smith B N (2002), Epilepsy Res 49:109-12.
76. Racine R J (1972), Electroencephalography and clinical neurophysiology 32(3):281-294.
77. Rao M S & Shetty A K (2004), Eur J Neurosci 19:234-246.

What is claimed is:

1. A medicament for inhibiting brain inflammation in an animal subsequent a brain injury comprising a preparation enriched for A1 exosomes, wherein said A1 exosomes have a mean size of about 85 nm to about 250 nm, are CD9−, and prevent an increase IL-6, IFNγ and I1-1β gene expression levels in an animal subsequent a brain injury.

2. The medicament of claim 1 wherein the A1 exosomes comprise about 15×10⁹ A1 exosomes.

3. The medicament of claim 1 wherein the brain injury is a status epilepticus episode.

4. A medicament for preserving memory recognition function in an animal subsequent to a brain function impairing disease, comprising a preparation enriched for A1 exosomes, wherein said A1 exosomes have a mean size of about 85 nm to about 250 nm, are CD9−, and prevents an increase IL-6, IFNγ and I1-1β gene expression levels in an animal subsequent a brain injury.

5. The medicament of claim 4 wherein the brain function impairing disease is status epilepticus, Alzheimer's disease or stroke.

6. An exosome preparation comprising an enriched population of A1 exosomes derived from mesenchymal stem cells, said A1 exosomes having the following characteristics:
a mean size of about 85 to about 250 nm;
a surface epitope that is CD9−; and
anti-inflammatory cytokine activity for preventing an increase in IL-6, IFNγ and I1-1B in an animal subsequent a brain injury.

7. The exosome preparation of claim 6 wherein said A1 exosomes are derived from a cell culture medium in which mesenchymal stem cells have been cultured.

8. A pharmaceutical preparation for treatment of brain inflammation comprising a therapeutically effective amount of the exosome preparation of claim 6 in a pharmaceutically acceptable carrier solution.

9. An intranasal pharmaceutical preparation comprising the pharmaceutical preparation of claim 8.

10. A medicament for treating Alzheimer's disease in an animal, comprising a pharmaceutical preparation comprising the exosome preparation of claim 6.

11. The medicament of claim 1 comprising an intra-nasally administered preparation.

12. The medicament of claim 7 wherein the mesenchymal stem cells are human mesenchymal stem cells.

13. The medicament claim 4 comprising an intra-nasally administered preparation.

14. The medicament claim 10 comprising an intra-nasally administered preparation.
